(19) United States
(12) Patent Application Publication
(10) Pub. No.: US 2013/0005726 A1
(43) Pub. Date: Jan. 3, 2013

(54) COMPOSITIONS AND METHODS FOR TREATING INFLAMMATORY DISORDERS

(76) Inventors: Derek Abbott, Shaker Heights, OH (US); Justine Tigno-Aranjuez, Shaker Heights, OH (US)

(21) Appl. No.: 13/583,869

(22) PCT Filed: Mar. 8, 2011
(86) PCT No.: PCT/US2011/027555

§ 371 Date: Sep. 10, 2012

Related U.S. Application Data
(60) Provisional application No. 61/311,591, filed on Mar. 8, 2010.
(60) Provisional application No. 61/408,827, filed on Nov. 1, 2010.

(51) Int. Cl. A61K 31/517; A61P 11/06; C12N 5/071; A61P 1/00; A61K 31/5377; A61P 29/00; A61P 37/06

(52) U.S. Cl. 514/234.5; 514/266.1; 514/266.4; 435/375

(57) ABSTRACT

A method for treating in a subject with an inflammatory disorder and/or immunological disorder associated with NOD2 activation includes administering to the subject a therapeutically effective amount of at least one tyro sine kinase inhibitor that substantially inhibits nucleotide-binding oligomerization domain containing 2 (NOD2):receptor-interacting protein 2 (RIP2) signaling in a NOD2-bearing cell and is not cytotoxic to the cell.

### RELATED APPLICATION

This application claims priority from U.S. Provisional Application Nos. 61/311,591, filed Mar. 8, 2010 and 61/408,827 filed Nov. 1, 2010, the subject matter of which are incorporated herein by reference in their entirety.

### TECHNICAL FIELD

This application relates to compositions and methods for treating inflammatory disorders, and more particularly to compositions and methods for treating inflammatory disorders associated with nucleotide-binding oligomerization domain containing 2 (NOD2) NOD2:RIP2 signaling as well as activation nuclear factor kappa-light-chain-enhancer of activated B cells (NFκB).

### BACKGROUND

Lack of coordination between inflammatory signaling pathways influences the development of inflammatory disorders, such as sacrcoidosis, rheumatoid arthritis, and inflammatory bowel disease. Inflammatory signal coordination can be modeled through the study of NLRP protein, NOD2. NOD2 was originally identified as the first Crohn's disease susceptibility gene. In the years since that discovery, NOD2 has been genetically linked to other inflammatory diseases, such as Blau Syndrome and Early Onset Sarcoidosis (EOS).

Crohn's disease affects 1 in 500/1000 Americans (approximately 440,000 people), and sarcoidosis affects approximately 154,000 Americans with the majority being African American. The card15 gene (coding for NOD2) is the most prevalent genetic polymorphism/mutation encountered in either of these patient populations.

Treatment for both of these disorders currently relies on broad, non-specific immunologic inhibition (e.g., corticosteroids) or on specific cytokine inhibition (e.g., anti-TNF therapies) with significant costs and side effects. Treatment is less than ideal, however, because not all agents are equally efficacious, the diseases occur over long time frames, and not all agents remain efficacious in the same patient.

### SUMMARY

This application relates to a method for treating a subject with an inflammatory disorder and/or immunological disorder associated with nucleotide-binding oligomerization domain containing 2 (NOD2) activation. The method includes administering to the subject a therapeutically effective amount of at least one tyrosine kinase inhibitor that substantially inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

Another aspect of the application relates to a method for treating an inflammatory disorder and/or immunological disorder associated with MDP-induced, NFκB activation in a subject. The method includes administering to the subject a therapeutically effective amount of at least one EGFR inhibitor. The at least one EGFR inhibitor inhibits RIP2 kinase activity and phosphorylation of RIP2 in a NOD2-bearing cell of the subject.

A further aspect relates to a method for treating inflammatory bowel disease in a subject associated with NOD2 activation. The method includes administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

A still further aspect relates to a method for treating Crohn's disease in a subject. The method includes administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

Yet another aspect relates to a method for treating sarcoidosis in a subject. The method includes administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

Another aspect relates to a method for treating Blau syndrome in a subject. The method includes administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

Yet another aspect relates to a method for treating asthma in a subject associated with aberrant NOD2 activation. The method includes administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell. The amount of tyrosine kinase inhibitor administered to the subject is not cytotoxic to the cell.

Another aspect of the application relates to a method for inhibiting MDP-induced, NFκB activation in a NOD2-bearing cell. The method includes administering to the NOD2-bearing cell an amount of at least one tyrosine kinase inhibitor that inhibits nucleotide-binding oligomerization domain containing 2 (NOD2):receptor-interacting protein 2 (RIP2) signaling in the cell, wherein the amount is not cytotoxic to the NOD2-bearing cell.

A further aspect of the application relates to a pharmaceutical composition for treating an inflammatory disorder and/or immunological disorder associated with nucleotide-binding oligomerization domain containing 2 (NOD2) activation. The pharmaceutical composition can include a therapeutically effective amount of at least one tyrosine kinase inhibitor that substantially inhibits nucleotide-binding oligomerization domain containing 2 (NOD2):receptor-interacting protein 2 (RIP2) signaling in a NOD2-bearing cell. The amount of tyrosine kinase inhibitor is not cytotoxic to the cell.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a western blot showing MDP inducible RIP2 tyrosine phosphorylation in HT-29 cells stimulated with MDP.

FIG. 2 illustrates a western blot showing RIP2 tyrosine phosphorylation of HEK293 cells upon transfection with RIP2 and wild-type NOD2 or NOD2 deletion mutants except for NOD2 lacking the CARD domains.

FIG. 3 illustrates a western blot showing RIP2 tyrosine phosphorylation of HEK293 cells upon transfection with RIP2 and wild-type NOD2, but not with Crohn's disease associated NOD2 polymorphism L1007insC.

FIG. 4 illustrates a western blot showing purification of tyrosine phosphorylated RIP2 which was sent for mass spectrometry as well as the mass spectrometric information about the discovered phosphorylation sites.

FIG. 5 illustrates a western blot showing HA-tagged NOD2 coprecipitates with RIP2, Y474F RIP2, Y520F RIP2, or Y474FY520F RIP2 after coexpression from HEK293 cells.

FIG. 6 illustrates a western blot showing RIP2 tyrosine phosphorylation when HEK293 cells are transfected with NOD2 in the presence of wild type (WT) or Y520F RIP2 but not Y474F RIP2 confirming the site of NOD2-induced RIP2 tyrosine phosphorylation.

FIG. 7 illustrates a graph showing NFκB activation levels of HEK293 cells expressing WT RIP2, Y474F RIP2, Y520F RIP2, or Y474F Y520F RIP2.

FIG. 8 illustrates a graph showing NFκB activation levels of RIP^{-/-}MEF cells expressing NOD2, NOD2+RIP2, or NOD2+Y474F RIP2.

FIG. 9 illustrates a graph showing NFκB activation levels of HEK293 cells transfected with NOD2, NOD2+RIP2+siRNA against endogenous RIP2, or NOD2+Y474F RIP2+siRNA against endogenous RIP2.

FIG. 10 illustrates a western blot showing RIP2 tyrosine phosphorylation of HEK293 cells transfected with NOD2 in the presence of wild-type or K47A (kinase inactive) RIP2.

FIG. 11 illustrates a western blot showing an in vitro kinase assay performed in the presence or absence of ATP using WT or K47A RIP2 immunoprecipitated from HEK293 cells transfected with RIP2, K47A RIP2, NOD2, or NOD2.

FIG. 12 illustrates a western blot showing RIP2 tyrosine phosphorylation in HEK293 cells transfected with RIP2, IKKβ, and NOD2 and treated with 10 nM, 100 nM, or 1 μM of erlotinib or gefitinib.

FIG. 13 illustrates a western blot showing tryrosine phorphorylation, serine phosphorylation, and total phosphorylation of immunoprecipitated RIP2 or K47A RIP2 subjected to an in vitro kinase assay in the presence of erlotinib or gefitinib.

FIG. 14 illustrates a western blot showing RIP2 tyrosine phosphorylation in HEK293 cells transfected with NOD2, and wild type or T95M RIP2 treated with 100 nM, 500 nM, or 2 μM of erlotinib or gefitinib.

FIG. 15 illustrates a western blot showing MDP-inducible RIP2 tyrosine phosphorylation in HT-29 cells overexpressing NOD2, and either treated or not treated with erlotinib or gefitinib before stimulation with MDP.

FIG. 16 illustrates a western blot showing MDP-inducible RIP2 tyrosine phosphorylation of RAW264.7 macrophages overexpressing NOD2, and either treated or not treated with erlotinib or gefitinib before stimulation with MDP.

FIG. 17 illustrates western blots showing tyrosine phosphorylation of HT-29 cells treated with increasing doses of erlotinib or gefitinib (10 nM, 100 nM, 1 μM) before treatment with MDP.

FIG. 18 illustrates a graph showing IL-6 expression of RAW264.7 macrophages overexpressing NOD2 or the activating R334Q NOD2 mutation associated with Blau Syndrome pretreated with erlotinib or gefitinib before stimulation with MDP or MDP and LPS.

FIG. 19 illustrates images showing graphs show IL-6 and G-SCF expression in response to MDP of bronchoalvelolar lavage (BA) samples treated with erlotinib or gefitinib from two human patients with active sarcoidosis.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which this application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application.

As used herein, the term “activity” with reference to nuclear factor kappa-light-chain-enhancer of activated B cells (NFκB) activity can refer to a cellular, biological, and/or therapeutic activity or function of NFκB. Examples of such activities can include, but are not limited to, signal transduction, interacting or associating with DNA or other binding partner(s) or cellular component (s), and modulating cellular responses to stimuli, such as stress, cytokines, free radicals, UV radiation, oxidized LDL, and bacterial or viral antigens.

As used herein, the terms “inflammatory disorder” or “inflammatory disease” can refer to a disorder or disease characterized by aberrant activation of the immune system that leads to or causes pathogenesis of several acute and chronic conditions including, for example, sarcoidosis, rheumatoid arthritis, inflammatory bowel disease, transplant rejection, colitis, gastritis and ileitis. An inflammatory disease can include a state in which there is a response to tissue damage, cell injury, an antigen, an infectious disease, and/or some unknown cause. Symptoms of inflammation may include, but are not limited to, cell infiltration and tissue swelling.

As used herein, the term “subject” can refer to any animal, including, but not limited to, humans and non-human animals (e.g., rodents, arthropods, insects, fish), non-human primates, ovines, bovines, ruminants, lagomorphs, porcines, caprines, equines, canines, felines, and ayes.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

As used herein, the term “therapeutically effective amount” can refer to that amount of one or more agents (e.g., a tyrosine kinase inhibitor) that result in amelioration of inflammatory disease symptoms or a prolongation of survival in a subject. A therapeutically relevant effect relieves to some extent one or more symptoms of an inflammatory disease or returns to normal, either partially or completely, one or more physiological or biochemical parameters associated with or causative of the disease.

As used herein, the term “polypeptide” can refer to an oligopeptide, peptide, polypeptide, or protein sequence, or to a fragment, portion, or subunit of any of these, and to naturally occurring or synthetic molecules. The term “polypeptide” can also include amino acids joined to each other by peptide bonds or modified peptide bonds, i.e., peptide isosteres, and may contain any type of modified amino acids. The term “polypeptide” can also include peptides and polypeptide fragments, motifs and the like, glycosylated polypeptides, and all “mimetic” and “peptidomimetic” polypeptide forms.

As used herein, the term “polynucleotide” can refer to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material natural or synthetic in origin, including, e.g., iRNA, siRNA, microRNA, ribonucleoproteins (e.g., iRNPs). The term can also encompass nucleic acids, i.e., oligonucleotides, containing known analogues of natural nucleotides. Additionally, the term can encompass nucleic acid-like structures with synthetic backbones.

As used herein, the term “wild type” refers to the naturally-occurring polynucleotide sequence encoding a protein, or a portion thereof, or protein sequence, or portion thereof, respectively, as it normally exists in vivo.

As used herein, the term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

As used herein, the phrases “parenteral administration” and “administered parenterally” means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the phrases “systemic administration,” “administered systemically,” “peripheral administration” and “administered peripherally” mean the administration of a compound, drug or other material other than directly into the central nervous system, such that it enters the animal's system and, thus, is subject to metabolism and other like processes, for example, subcutaneous administration.

This application relates to compositions and methods for treating inflammatory disorders (or diseases) and/or immunological disorders (or diseases) associated with nucleotide-binding oligomerization domain containing 2 (NOD2) activation, NOD2:protein kinase receptor-interacting protein 2 (NOD2:RIP2) signaling and/or muramyl dipeptide (MDP) induced NFκB activation. NOD2 is an intracellular protein that is activated upon exposure to a bacterial breakdown product, muramyl dipeptide (MDP). Upon MDP exposure, NOD2 binds RIP2 to form the functional NOD2:RIP2 signaling complex. The NOD2:RIP2 complex then induces the K63 (lysine-63) linked polyubiquitination of the IKK scaffolding protein NEMO to initiate NFκB activation.

It was found that upon NOD2 activation by exposure to MDP, RIP2 is tyrosine phosphorylated at tyrosine-474 (Y474). Phosphorylation of this tyrosine is necessary for maximal RIP-2 induced NFκB activation, maximal signaling synergy with NOD2, and activation of other pathways downstream of NOD2:RIP2. It was also found that RIP2 possesses tyrosine kinase activity and is capable of autophosphorylation in response to NOD2 activation. It was further found that tyrosine kinase inhibitors that can inhibit RIP2 kinase activity can inhibit both Y474 RIP2 phosphorylation and that this inhibition can dampen or inhibit NOD2:RIP2 signaling complex activation of NFκB and other pathways downstream of NOD2:RIP2. Inhibition of NOD2:RIP2 signaling can be used treat inflammatory disorder and immunological disorders in which NOD2 is overactive.

An aspect of the application therefore relates to a method of treating inflammatory disorders (or diseases) and/or immunological disorders (or diseases) associated with NOD2 activation, NOD2:RIP2 signaling, and/or muramyl dipeptide (MDP) induced NFκB activation (and activation of other pathways downstream of NOD2:RIP2), by administering a therapeutically effective amount of an agent to the subject that inhibits phosphorylation of RIP2, thereby preventing formation of the fully-activated NOD2:RIP2 complex and inhibiting activation of NFκB and other pathways downstream of NOD2:RIP2.

Generally, the inflammatory disorder and/or immunological disorder treated by the methods described herein can include any condition, disease, or disorder where the NFκB signal transduction pathway and/or NFκB activity in a cell of the subject can be modulated (e.g., decreased or inhibited) and/or where the inflammatory disorder results from other pathways downstream of NOD2:RIP2. Examples of cells in which the NFκB signal transduction pathway and/or NFκB activity can be modulated include immune cells, such as leukocytes, monocytes, and macrophages and epithelial cells, such as enterocytes, colonic epithelial cells, respiratory epithelial cells or keratinocytes (NOD2 bearing cells or cells in which NOD2 can be upregulated).

In some embodiments, the inflammatory disease and/or immunological disorder can be selected from the group consisting of achlorhydra autoimmune active chronic hepatitis, acute disseminated encephalomyelitis, acute hemorrhagic leukoencephalitis, Addison's disease, agammaglobulinemia, alopecia greata, Alzheimer's disease, amyotrophic lateral sclerosis, ankylosing spondylitis, anti-gbm/tbm nephritis, antiphospholipid syndrome, antisynthetase syndrome, aplastic anemia, arthritis, asthma, atopic allergy, atopic dermatitis, autoimmune cardiomyopathy, autoimmune hemolytic anemia, autoimmune hepatitis, autoimmune inner ear disease, autoimmune lymphoproliferative syndrome, autoimmune peripheral neuropathy, autoimmune polyendocrine syndrome, autoimmune progesterone dermatitis, autoimmune thrombocytopenia purpura, autoimmune uveitis, balo disease/balo concentric sclerosis, Bechets syndrome, Berger's disease, Bickerstaff's encephalitis, Blau syndrome, bullous pemphigoid, Castleman's disease, Chagas disease, chronic fatigue immune dysfunction syndrome, chronic inflammatory demyelinating polyneuropathy, chronic lyme disease, chronic obstructive pulmonary disease, Churg-Strauss syndrome, cicatricial pemphigoid, coeliac disease, Cogan syndrome, cold agglutinin disease, colitis, cranial arteritis, crest syndrome, Crohns disease, Cushing's syndrome, Dego's disease, Dercum's disease, dermatitis herpetiformis, dermatomyositis, diabetes mellitus type 1, Dressler's syndrome, discoid lupus erythematosus, eczema, endometriosis, enthesitis-related arthritis, eosinophilic fasciitis, epidermolysis bullosa acquisita, essential mixed cryoglobulinemia, Evan's syndrome, fibrodysplasia ossificans progressive, fibromyalgia, fibromyositis, fibrosing aveolitis, gastritis, gastrointestinal pemphigoid, giant cell arteritis, glomerulonephritis, Goodpasture's syndrome, graft versus host disease, Graves' disease, Guillain-barré syndrome (GBS), Hashimoto's encephalitis, Hashimoto's thyroiditis, henoch-schonlein purpura, hidradenitis suppurativa, Hughes syndrome, inflammatory bowel disease (IBD), idiopathic inflammatory demyelinating diseases, idiopathic pulmonary fibrosis, idiopathic thrombocytopenic purpura, iga nephropathy, inflammatory demyelinating polyneuopathy, interstitial cystitis, irritable bowel syndrome (IBS), Kawasaki's disease, lichen planus, Lou Gehrig's disease, lupoid hepatitis, lupus erythematosus, ménière's disease, microscopic polyangiitis, mixed connective tissue disease, morphea, multiple myeloma, multiple sclerosis, myasthenia gravis, myositis, narcolepsy, neuromyelitis optica, neuromyotonia, occular cicatricial pemphigoid, opsoclonus myoclonus syndrome, ord thyroiditis, Parkinson's disease, pars planitis, pemphigus, pemphigus vulgaris, pernicious anaemia, polymyalgia rheumatic, polymyositis, primary biliary cirrhosis, primary sclerosing cholangitis, progressive inflammatory neuropathy, psoriasis, psoriatic arthritis, raynaud phenomenon, relapsing polychondritis, Reiter's syndrome, rheumatoid arthritis, rheumatoid fever, sarcoidosis, schizophrenia, Schmidt syndrome, Schnitzler syndrome, scleritis, scleroderma, Sjögren's syndrome, spondyloarthropathy, sticky blood syndrome, still's disease, stiff person syndrome, sydenham chorea, sweet syndrome, takayasu's arteritis, temporal arteritis, transverse myelitis, ulcerative colitis, undifferentiated connective tissue disease, undifferentiated spondyloarthropathy, vasculitis, vitiligo, Wegener's granulomatosis, Wilson's syndrome, Wiskott-Aldrich syndrome, hypersensitivity reactions of the skin, atherosclerosis, ischemia-reperfusion injury, myocardial infarction, and restenosis.

In other embodiments, the inflammatory disease can include any condition, disease, or disorder associated with bacterial breakdown product-induced, NFκB activation. Examples of bacterial breakdown products can include MDP and lipopolysaccharide (LPS). Inflammatory disorders associated with MDP-induced, NFκB activation can include, for example, sarcoidosis (e.g., Early Onset Sarcoidosis or EOS), Blau Syndrome, inflammatory bowel disease (IBD) (e.g., Crohn's disease and ulcerative colitis), rheumatoid arthritis, colitis, gastritis, ileitis, asthma, and/or graft versus host disease.

The agent administered to the subject with the inflammatory and/or immunological disorder can include a small molecule, polypeptide, polynucleotide, other therapeutic composition, or combination thereof that is capable of decreasing or inhibiting phosphorylation of RIP2, RIP2 kinase activity, NOD2:RIP2 signaling, and/or NOD2:RIP2 complex activation of NFκB and other pathways downstream of NOD2:RIP2 in the NOD2-bearing cell without being cytoxic to the cell at therapeutically effective amounts. In one aspect, the agent can include a small molecule, polypeptide, polynucleotide, other therapeutic composition, or combination thereof which is capable of inhibiting phosphorylation of RIP2 (e.g., by inhibiting phosphorylation of Y474 RIP2). By inhibiting phosphorylation of RIP2, it is meant reducing phosphorylation of RIP2 in a NOD2-bearing cell, such as a leukocyte, upon NOD2 activation by at least about 10%, at least about 20%, at least about 30%, at least about 40%, at least about 50%, at least about 60%, at least about 70%, at least about 80%, at least about 90%, or at least about 95% compared to an untreated NOD2 activated leukocyte.

In an aspect of the application, an agent that is capable of inhibiting phosphorylation of RIP2 can include a tyrosine kinase inhibitor that is capable of decreasing or inhibiting RIP2 kinase activity and/or phosphorylation of RIP2. For example, the tyrosine kinase inhibitor can include a small molecule, polypeptide, polynucleotide, other therapeutic composition, or combination thereof which is capable of inhibiting the activity of the RIP2 kinase responsible for phosphorylating Y474 RIP2. Alternatively, the tyrosine kinase inhibitor can include a small molecule, polypeptide, polynucleotide, other therapeutic composition, or combination thereof which is capable of interacting with RIP2 so as to block (e.g., sterically block) or hinder addition of a phosphate group to Y474 RIP2. In some embodiments, the tyrosine kinase inhibitor, when administered at a therapeutically effective amount to a NOD2-bearing cell of subject being treated, can substantially inhibit RIP2 kinase in the NOD2-bearing cell (e.g., macrophage) to which it is administered without being cytoxic to the cell.

In an embodiment of the application, a tyrosine kinase inhibitor that inhibits RIP2 kinase activity can include an epidermal growth factor receptor (EGFR) inhibitor. By “EGFR inhibitor”, it is meant an agent that inhibits EGFR tyrosine kinase by binding to the adenosine triphosphate (ATP)-binding site of the enzyme. It was found that tyrosine kinase inhibitors that are effective at selectively inhibiting the kinase activity of EGFR are also effective at inhibiting RIP2 kinase activity and RIP2 autophosphorylation of Y474 of RIP2. In an aspect of the application, the EGFR inhibitor can substantially inhibit RIP2 kinase in the immune cells (e.g., macrophage) or epithelial cell (e.g., Colonic epithelial cell) to which it is administered at nanomolar concentrations (e.g., about 10 nm to about 500 nm) without being cytoxic to the cell. In another aspect, the EGFR inhibitor can be as effective or more effective at inhibiting RIP2 kinase activity as inhibiting EGFR kinase activity.

In an embodiment of the application, the EGFR inhibitor can be a quinazoline derivative of the formula I:

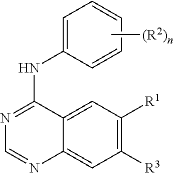

wherein
n is 1, 2 or 3 and each R² is independently halogeno, trifluoromethyl or (1-4C)alkyl;
R³ is (1-4C)alkoxy; and
R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy,
and wherein any of the above-mentioned R¹ substituents comprising a CH₂ (methylene) group which is not attached to a N or O atom optionally bears on said CH₂ group a hydroxy substituent;
or a pharmaceutically-acceptable salt thereof.

In this specification the term “alkyl” includes both straight and branched chain alkyl groups but references to individual alkyl groups such as “propyl” are specific for the straight chain version only. For example when R¹ is a di-[(1-4C)alkyl]amino-(2-4C)alkoxy group, suitable values for this generic radical include 2-dimethylaminoethoxy, 3-dimethylaminopropoxy, 2-dimethylaminopropoxy and 1-dimethylaminoprop-2-yloxy. An analogous convention applies to other generic terms.

Within the specification it is to be understood that, insofar as certain of the compounds of the formula I may exist in optically active or racemic forms by virtue of one or more substituents containing an asymmetric carbon atom, the invention encompasses any such optically active or racemic form which possesses anti-proliferative activity. The synthesis of optically active forms may be carried out by standard techniques of organic chemistry well known in the art, for example by synthesis from optically active starting materials or by resolution of a racemic form. It is also to be understood that certain quinazoline derivatives of the formula I can exist in solvated as well as unsolvated forms such as, for example, hydrated forms.

An example of a pharmaceutically-acceptable salt of a quinazoline derivative of the application is an acid-addition salt of a quinazoline derivative, which is sufficiently basic, for example, a mono- or di-acid-addition salt with, for example, an inorganic or organic acid, for example hydrochloric, hydrobromic, sulphuric, phosphoric, trifluoroacetic, citric, maleic, tartaric, fumaric, methanesulphonic or 4-toluenesulphonic acid.

In some embodiments, EGFR inhibitors include quinazoline derivatives of the formula I, or pharmaceutically-acceptable salts thereof, wherein:
(a) n is 1 or 2 and each R² is independently fluoro, chloro, bromo, trifluoromethyl or methyl; and R³ and R¹ have any of the meanings defined hereinbefore;
(b) n is 1, 2 or 3 and each R² is independently fluoro, chloro or bromo; and R³ and R¹ have any of the meanings defined hereinbefore;
(c) R³ is methoxy or ethoxy; and n, R² and R¹ have any of the meanings defined hereinbefore;
(d) R¹ is 2-dimethylaminoethoxy, 2-diethylaminoethoxy, 3-dimethylaminopropoxy, 3-diethylaminopropoxy, 2-(pyrrolidin-1-yl)ethoxy, 3-(pyrrolidin-1-yl)propoxy, 2-piperidinoethoxy, 3-piperidinopropoxy, 2-morpholinoethoxy, 3-morpholinopropoxy, 2-(piperazin-1-yl)ethoxy, 3-(piperazin-1-yl)propoxy, 2-(4-methylpiperazin-1-yl)ethoxy, 3-(4-methylpiperazin-1-yl)propoxy, 2-(imidazol-1-yl)ethoxy, 3-(imidazol-1-yl)propoxy, 2-[di-(2-methoxyethyl)amino]ethoxy, 3-[di-(2-methoxyethyl)amino]propoxy, 3-dimethylamino-2-hydroxypropoxy, 3-diethylamino-2-hydroxypropoxy, 3-(pyrrolidin-1-yl)-2-hydroxypropoxy, 3-piperidino-2-hydroxypropoxy, 3-morpholino-2-hydroxypropoxy, 3-(piperazin-1-yl)-2-hydroxypropoxy or 3-(4-methylpiperazin-1-yl)-2-hydroxypropoxy;
and n, R² and R³ have any of the meanings defined hereinbefore;
(e) R¹ is 3-dimethylaminopropoxy, 3-diethylaminopropoxy, 3-(pyrrolidin-1-yl)propoxy, 3-piperidinopropoxy, 3-morpholinopropoxy, 3-(piperazin-1-yl)propoxy, 3-(4-methylpiperazin-1-yl)propoxy, 3-(imidazol-1-yl)propoxy, 3-[di-(2-methoxyethyl)amino]propoxy, 3-dimethylamino-2-hydroxypropoxy, 3-diethylamino-2-hydroxypropoxy, 3-(pyrrolidin-1-yl)-2-hydroxypropoxy, 3-piperidino-2-hydroxypropoxy, 3-morpholino-2-hydroxypropoxy, 3-(piperazin-1-yl)-2-hydroxypropoxy or 3-(4-methylpiperazin-1-yl)-2-hydroxypropoxy;
and n, R² and R³ have any of the meanings defined hereinbefore or in this section relating to particular novel compounds of the invention;
(f) R¹ is 3-dimethylaminopropoxy, 3-diethylaminopropoxy, 3-(pyrrolidin-1-yl)propoxy, 3-morpholinopropoxy or 3-morpholino-2-hydroxypropoxy; and n, R² and R³ have any of the meanings defined hereinbefore or in this section relating to particular compounds;
(g) R¹ is 3-morpholinopropoxy; and n, R² and R³ have any of the meanings defined hereinbefore or in this section relating to particular compounds.

Examples of quinazoline derivatives of formula I are 4-(3′-chloro-4′-fluoroanilino)-7-methoxy-6-(2-pyrrolidin-1-ylethoxy)quinazoline; 4-(3′-chloro-4′-fluoroanilino)-7-methoxy-6-(2-morpholinoethoxy)quinazoline; 4-(3′-chloro-4′-fluoroanilino)-7-methoxy-6-[2-(4-methylpiperazin-1-yl)ethoxy]quinazoline; 4-(3′-chloro-4′-fluoroanilino)-7-methoxy-6-{2-[di-(2-methoxyethyl)amino]ethoxy}quinazoline; 4-(3′-chloro-4′-fluoroanilino)-6-(2-dimethylaminoethoxy)-7-methoxyquinazoline; 4-(3′-chloro-4′-fluoroanilino)-6-(2-diethylaminoethoxy)-7-methoxyquinazoline; 4-(2′,4′-difluoroanilino)-6-(3-dimethylaminopropoxy)-7-methoxyquinazoline; 4-(3′-chloro-4′-fluoroanilino)-6-(2-hydroxy-3-morpholinopropoxy)-7-methoxyquinazoline; 4-(2′,4′-difluoroanilino)-7-methoxy-6-(3-morpholinopropoxy)quinazoline; 4-(3′-chloro-4′-fluoroanilino)-6-(2-imidazol-1-ylethoxy)-7-methoxyquinazoline; 4-(3′-chloro-4′-fluoroanilino)-6-(3-diethylaminopropoxy)-7-methoxyquinazoline; 4-(3′-chloro-4′-fluoroanilino)-7-methoxy-6-(3-pyrrolidin-1-ylpropoxy)quinazoline; 4-(3′-chloro-4′-fluoroanilino)-6-(3-dimethylaminopropoxy)-7-methoxyquinazoline; 4-(3′,4′-difluoroanilino)-6-(3-dimethylaminopropoxy)-7-methoxyquinazoline; 4-(3′,4′-difluoroanilino)-7-methoxy-6-(3-morpholinopropoxy)quinazoline; 6-(3-diethylaminopropoxy)-4-(3′,4′-difluoroanilino)-7-methoxyquinazoline; 4-(3′-chloro-4′-fluoroanilino)-7-methoxy-6-(3-piperidinopropoxy)quinazoline; 4-(3′-chloro-4′-fluoroanilino)-7-methoxy-6-(2-piperidinoethoxy)quinazoline; 4-(3′-chloro-4′-fluoroanilino)-6-(3-imidazol-1-ylpropoxy)-7-methoxyquinazoline; 4-(3′-chloro-4′-fluoroanilino)-7-methoxy-6-(3-morpholinopropoxy)quinazoline; 4-(3′-chloro-4′-fluoroanilino)-7-methoxy-6-(3-morpholinopropoxy)quinazoline; 4-(3′-chloro-4′-fluoroanilino)-7-methoxy-6-(3-morpholinopropoxy)quinazoline.

A quinazoline derivative of formula I, or a pharmaceutically-acceptable salt thereof, may be prepared by any process known to be applicable to the preparation of chemically-related compounds. Examples of processes include those illustrated in U.S. Pat. Nos. 5,457,105, 5,616,582, and 5,770,599 as well as European Patent Applications Nos. 0520722, 0566226, 0602851, 0635498 and 0635507, which are incorporated herein by reference in their entirety. Necessary starting materials may be obtained by standard procedures of organic chemistry. Alternatively necessary starting materials are obtainable by analogous procedures to those illustrated which are within the ordinary skill of an organic chemist.

In other embodiments, the EGFR inhibitor can be a quinazoline derivative of the formula II:

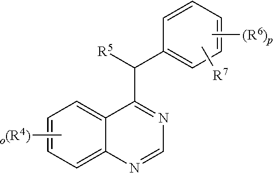

and to pharmaceutically acceptable salts and prodrugs thereof, wherein:
o is 1, 2, or 3;
each R⁴ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R⁴ is independently selected from R¹² and (C₁-C₄)-alkyl substituted by cyano, wherein R¹² is selected from the group consisting of R⁸, —OR⁹, —NR⁹ R⁹, —C(O)R¹⁰, —NHOR⁸, —OC(O)R⁹, cyano, A and —YR⁸; R⁸ is C₁-C₄ alkyl; R⁹ is independently hydrogen or R⁸; R¹⁰ is R⁸, —OR⁹ or —NR⁹ R⁹; A is selected from piperidino, morpholino, pyrrolidino, 4-R⁹-piperazin-1-yl, imidazol-1-yl, 4-pyridon-1-yl, —(C₁-C₄ alkylene)(CO₂ H), phenoxy, phenyl, phenylsulfanyl, C₂-C₄ alkenyl, and —(C₁-C₄ alkylene)C(O)NR⁹ R⁹; and Y is S, SO, or SO₂; wherein the alkyl moieties in R⁸, —OR⁹ and —NR⁹ R⁹ are optionally substituted by one to three substituents independently selected from halo and R¹², and wherein the alkyl moieties of said optional substituents are optionally substituted by halo or R¹², with the proviso that two heteroatoms are not attached to the same carbon atom, and with the further proviso that no more than three R¹² groups may comprise a single R⁴ group;
or each R⁴ is independently selected from —NHSO₂ R⁸, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹³—(C₂-C₄)-alkanoylamino wherein R¹³ is selected from halo, —OR⁹, C₂-C₄ alkanoyloxy, —C(O)R¹⁰, and —NR⁹ R⁹; and wherein the foregoing R⁴ groups are optionally substituted by 1 or 2 substituents independently selected from halo, C₁-C₄ alkyl, cyano, methanesulfonyl and C₁-C₄ alkoxy;
or two R⁴ groups are taken together with the carbons to which they are attached to form a 5-8 membered ring that includes 1 or 2 heteroatoms selected from O, S and N;
R⁵ is hydrogen or C₁-C₆ alkyl optionally substituted by 1 to 3 substituents independently selected from halo, C₁-C₄ alkoxy, —NR⁹ R⁹, and —SO₂ R⁸;
p is 1 or 2 and each R³ is independently selected from hydrogen, halo, hydroxy, C₁-C₆ alkyl, —NR⁹ R⁹, and C₁-C₄ alkoxy, wherein the alkyl moieties of said R⁶ groups are optionally substituted by 1 to 3 substituents independently selected from halo, C₁-C₄ alkoxy, —NR⁹ R⁹, and —SO₂ R⁸; and,
R⁷ is azido or -(ethynyl)-R¹⁴ wherein R¹⁴ is hydrogen or C₁-C₆ alkyl optionally substituted by hydroxy, —OR⁹, or —NR⁹ R⁹.

In some embodiments, compounds of formula II include those wherein R⁵ is hydrogen and R⁷ is -(ethynyl)-R¹⁴.

In other embodiments, compounds of formula II include those wherein m is 1 or 2;
each R⁴ is independently selected from the group consisting of hydrogen, hydroxy, hydroxyamino, carboxy, nitro, carbamoyl, ureido, R⁸ optionally substituted with halo, —OR⁹, carboxy, —C(O)NR⁹ R⁹, A or —NR⁹ R⁹; —OR⁸ optionally substituted with halo, —OR⁹, —OC(O)R⁹, —NR⁹ R⁹, or A; —NR⁹ R⁹, —C(O)R⁹ R⁸, —SR⁸, phenyl-(C₂-C₄)-alkoxy, cyano, phenyl; —NHR⁸ optionally substituted with halo or R¹² wherein said R¹² is optionally substituted by R¹²; —NHOR⁸, —SR⁸, C₁-C₄ alkylsulfonylamino, phthalimido-(C₁-C₄)-alkylsulfonylamino, 3-phenylureido, 2-oxopyrrolidin-1-yl, 2,5-dioxopyrrolidin-1-yl, halo-(C₂-C₄)-alkanoylamino, hydroxy-(C₂-C₄)-alkanoylamino, (C₂-C₄)-alkanoyloxy-(C₂-C₄)-alkanoylamino, (C₁-C₄)-alkoxy-(C₂-C₄)-alkanoylamino, (C₁-C₄)-alkoxycarbonyl-(C₂-C₄)-alkanoylamino, carbamoyl-(C₂-C₄)-alkanoylamino, N—(C₁-C₄)-alkylcarbamoyl-(C₂-C₄)-alkanoylamino, N,N-di-[(C₁-C₄)-alkyl]carbamoyl-(C₂-C₄)-alkanoylamino, amino-(C₂-C₄)-alkanoylamino, (C₁-C₄)-alkyl-amino-(C₂-C₄)-alkanoylamino, and di-(C₁-C₄)-alkyl-amino-(C₂-C₄)-alkanoylamino, and wherein said phenyl or phenoxy or anilino substituent in the foregoing R⁴ groups is optionally substituted with one or two substituents independently selected from halo, C₁-C₄ alkyl and C₁-C₄ alkoxy;
each R⁶ is independently selected from hydrogen, methyl, ethyl, amino, halo and hydroxy; and,
R⁷ is ethynyl.

Other compounds of formula II include those wherein each R⁴ is independently selected from hydrogen, hydroxy, hydroxyamino, nitro, carbamoyl, ureido, R⁸ optionally substituted with halo, —OR⁹, carboxy, or —C(O)NH₂; —OR⁸ optionally substituted with halo, —OR⁹, —OC(O)R⁹, —NR⁹ R⁹, or A; —NR⁹ R⁹, —C(O)NR⁹ R⁹, —SR⁸, phenyl-(C₂-C₄)-alkoxy wherein said phenyl moiety is optionally substituted with 1 or 2 substituents independently selected from halo, R⁸ or —OR⁸.

Other compounds of formula II include those wherein R⁵ is hydrogen and R⁷ is azido.

Still other compounds of formula II include those wherein R⁶ is halo and R⁴ is hydrogen or —OR⁸.

Other compounds of formula II include those wherein R⁴ is methoxy.

Still other compounds of formula II include the following: (6,7-dimethoxyquinazolin-4-yl)-(3-ethynylphenyl)-amine; (6,7-dimethoxyquinazolin-4-yl)-[3-(3′-hydroxypropyn-1-yl)phenyl]-amine; [3-(2′-(aminomethyl)-ethynyl)phenyl]-(6,7-dimethoxyquinazolin-4-yl)-amine; (3-ethynylphenyl)-(6-nitroquinazolin-4-yl)-amine; (6,7-dimethoxyquinazolin-4-yl)-(4-ethynylphenyl)-amine; (6,7-dimethoxyquinazolin-4-yl)-(3-ethynyl-2-methylphenyl)-amine; (6-aminoquinazolin-4-yl)-(3-ethynylphenyl)-amine; (3-ethynylphenyl)-(6-methanesulfonylaminoquinazolin-4-yl)-amine; (3-ethynylphenyl)-(6,7-methylenedioxyquinazolin-4-yl)-amine; (6,7-dimethoxyquinazolin-4-yl)-(3-ethynyl-6-methylphenyl)-amine; (3-ethynylphenyl)-(7-nitroquinazolin-4-yl)-amine; (3-ethynylphenyl)-[6-(4′-toluenesulfonylamino)quinazolin-4-yl]-amine; (3-ethynylphenyl)-{6-[2′-phthalimido-eth-1′-yl-sulfonylamino]quinazolin-4-yl}-amine; (3-ethynylphenyl)-(6-guanidinoquinazolin-4-yl)-amine; (7-aminoquinazolin-4-yl)-(3-ethynylphenyl)-amine; (3-ethynylphenyl)-(7-methoxyquinazolin-4-yl)-amine; (6-carbomethoxyquinazolin-4-yl)-(3-ethynylphenyl)-amine; (7-carbomethoxyquinazolin-4-yl)-(3-ethynylphenyl)-amine; [6,7-bis(2-methoxyethoxy)quinazolin-4-yl]-(3-ethynylphenyl)-amine; (3-azidophenyl)-(6,7-dimethoxyquinazolin-4-yl)-amine; (3-azido-5-chlorophenyl)-(6,7-dimethoxyquinazolin-4-yl)-amine; (4-azidophenyl)-(6,7-dimethoxyquinazolin-4-yl)-amine; (3-ethynylphenyl)-(6-methansulfonyl-quinazolin-4-yl)-amine; (6-ethansulfanyl-quinazolin-4-yl)-(3-ethynylphenyl)-amine; (6,7-dimethoxy-quinazolin-4-yl)-(3-ethynyl-4-fluoro-phenyl)-amine; (6,7-dimethoxy-quinazolin-4-yl)-[3-(propyn-1′-yl)-phenyl]-amine; [6,7-bis-(2-methoxy-ethoxy)-quinazolin-4-yl]-(5-ethynyl-2-methyl-phenyl)-amine; [6,7-bis-(2-methoxy-ethoxy)-quinazolin-4-yl]-(3-ethynyl-4-fluoro-phenyl)-amine, [6,7-bis-(2-chloro-ethoxy)-quinazolin-4-yl]-(3-ethynyl-phenyl)-amine; [6-(2-chloro-ethoxy)-7-(2-methoxy-ethoxy)-quinazolin-4-yl]-(3-ethynyl-phenyl)-amine; [6,7-bis-(2-acetoxy-ethoxy)-quinazolin-4-yl]-(3-ethynyl-phenyl)-amine; 2-[4-(3-ethynyl-phenylamino)-7-(2-hydroxy-ethoxy)-quinazolin-6-yloxy]-ethanol; [6-(2-acetoxy-ethoxy)-7-(2-methoxy-ethoxy)-quinazolin-4-yl]-(3-ethynyl-phenyl)-amine; [7-(2-chloro-ethoxy)-6-(2-methoxy-ethoxy)-quinazolin-4-yl]-(3-ethynyl-phenyl)-amine; [7-(2-acetoxy-ethoxy)-6-(2-methoxy-ethoxy)-quinazolin-4-yl]-(3-ethynyl-phenyl)-amine; 2-[4-(3-ethynyl-phenylamino)-6-(2-hydroxy-ethoxy)-quinazolin-7-yloxy]-ethanol; 2-[4-(3-ethynyl-phenylamino)-7-(2-methoxy-ethoxy)-quinazolin-6-yloxy]-ethanol; 2-[4-(3-ethynyl-phenylamino)-6-(2-methoxy-ethoxy)-quinazolin-7-yloxy]-ethanol; [6-(2-acetoxy-ethoxy)-7-(2-methoxy-ethoxy)-quinazolin-4-yl]-(3-ethynyl-phenyl)-amine; (3-ethynyl-phenyl)-{6-(2-methoxy-ethoxy)-7-[2-(4methyl-piperazin-1-yl)-ethoxy]-quinazolin-4-yl}-amine; (3-ethynyl-phenyl)-[7-(2-methoxy-ethoxy)-6-(2-morpholin-4-yl)-ethoxy)-quinazolin-4-yl]-amine; (6,7-diethoxyquinazolin-1-yl)-(3-ethynylphenyl)-amine; (6,7-dibutoxyquinazolin-1-yl)-(3-ethynylphenyl)-amine; (6,7-diisopropoxyquinazolin-1-yl)-(3-ethynylphenyl)-amine; (6,7-diethoxyquinazolin-1-yl)-(3-ethynyl-2-methyl-phenyl)-amine; [6,7-bis-(2-methoxy-ethoxy)-quinazolin-1-yl]-(3-ethynyl-2-methyl-phenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; and 2-[4-(3-ethynyl-phenylamino)-6-(2-methoxy-ethoxy)-quinazolin-7-yloxy]-ethanol.

Other compounds of formula II include the following: (6,7-dipropoxy-quinazolin-4-yl)-(3-ethynyl-phenyl)-amine; (6,7-diethoxy-quinazolin-4-yl)-(3-ethynyl-5-fluoro-phenyl)-amine; (6,7-diethoxy-quinazolin-4-yl)-(3-ethynyl-4-fluoro-phenyl)-amine; (6,7-diethoxy-quinazolin-4-yl)-(5-ethynyl-2-methyl-phenyl)-amine; (6,7-diethoxy-quinazolin-4-yl)-(3-ethynyl-4-methyl-phenyl)-amine; (6-aminomethyl-7-methoxy-quinazolin-4-yl)-(3-ethynyl-phenyl)-amine; (6-aminomethyl-7-methoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-methoxy-quinazolin-4-yl)-(3-ethynylphenyl)-amine; (6-aminocarbonylmethyl-7-ethoxy-quinazolin-4-yl)-(3-ethynylphenyl)-amine; (6-aminocarbonylethyl-7-ethoxy-quinazolin-4-yl)-(3-ethynylphenyl)-amine; (6-aminocarbonylmethyl-7-isopropoxy-quinazolin-4-yl)-(3-ethynylphenyl)-amine; (6-aminocarbonylmethyl-7-propoxy-quinazolin-4-yl)-(3-ethynylphenyl)-amine; (6-aminocarbonylmethyl-7-methoxy-quinazolin-4-yl)-(3-ethynylphenyl)-amine; (6-aminocarbonylethyl-7-isopropoxy-quinazolin-4-yl)-(3-ethynylphenyl)-amine; (6-aminocarbonylethyl-7-propoxy-quinazolin-4-yl)-(3-ethynylphenyl)-amine; (6,7-diethoxyquinazolin-1-yl)-(3-ethynylphenyl)-amine; (3-ethynylphenyl)-[6-(2-hydroxy-ethoxy)-7-(2-methoxy-ethoxy)-quinazolin-1-yl]-amine; [6,7-bis-(2-hydroxy-ethoxy)-quinazolin-1-yl]-(3-ethynylphenyl)-amine; [6,7-bis-(2-methoxy-ethoxy)-quinazolin-1-yl]-(3-ethynylphenyl)-amine; (6,7-dimethoxyquinazolin-1-yl)-(3-ethynylphenyl)-amine; (3-ethynylphenyl)-(6-methanesulfonylamino-quinazolin-1-yl)-amine; and (6-amino-quinazolin-1-yl)-(3-ethynylphenyl)-amine.

A quinazoline derivative of the formula II, or a pharmaceutically-acceptable salt thereof, may be prepared by any process known to be applicable to the preparation of chemically-related compounds. Examples of processes include those illustrated in U.S. Pat. No. 5,747,498, which is incorporated herein by reference in its entirety. Necessary starting materials may be obtained by standard procedures of organic chemistry. Alternatively necessary starting materials are obtainable by analogous procedures to those illustrated which are within the ordinary skill of an organic chemist.

In still other embodiments, EGFR inhibitors that are capable of inhibiting RIP2 kinase activity can include erlotinib and/or gefitinib, which are commercially available from respectively Genentech and AstraZeneca under the tradenames Tarceva and Iressa. It was found that erlotinib and gefitinib can substantially inhibit RIP2 kinase in NOD2-bearing cells (e.g., macrophage or epithelial cells) to which it is administered at nanomolar concentrations (e.g., about 10 nm to about 500 nm) without being cytoxic to the cells.

The agent can be administered to the subject in a pharmaceutical composition at a therapeutically effective amount and for a period of time effective to deliver the agent to at least one NOD2-bearing cell (e.g., a macrophage) in which the NFκB signal transduction pathway, NFκB activity, and/or other pathways downstream of NOD2:RIP2 can be modulated. It will be appreciated that the at least one agent may additionally comprise a pharmaceutically acceptable carrier. Pharmaceutically acceptable carriers are known in the art, and may include any material or materials, which are not biologically or otherwise undesirable, i.e., the material may be incorporated or added into the agent without causing any undesirable biological effects or interacting in a deleterious manner with any of the other components of the composition. When the term “pharmaceutically acceptable” is used to refer to a pharmaceutical carrier, it can be implied that the carrier has met the required standards of toxicological and manufacturing testing or that it is included on the Inactive Ingredient Guide prepared by the U.S. Food and Drug administration.

Determination of a therapeutically effective amount is within the capability of those skilled in the art. The exact formulation, route of administration, and dosage can be chosen by a medical professional, for example, in view of the subject's condition. It should also be understood that a “therapeutically effective amount” may be interpreted as an amount giving a desired therapeutic effect, whether taken in one dose or in any number of doses, or taken alone or in combination with other agents. In the case of the present invention, a “therapeutically effective amount” may be understood as an amount of one or more of the agents described herein required to treat an inflammatory disorder, such as an inflammatory diseases associated with bacterial breakdown product-induced, activation of NFκB and other pathways downstream of NOD2:RIP2, in a subject.

The location(s) where the agent and/or composition is/are administered may be determined based on the subject's individual need, such as the particular type of inflammatory disorder. For example, the agent and/or composition may be systemically or parenterally, by being injected intravenously into the subject. It will be appreciated that other routes of administration may be used including, for example, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal routes.

Upon injection into the subject, the at least one agent can be delivered to at least one NOD2-bearing cell (e.g., a macrophage) or epithelial cell in which the NFκB signal transduction pathway, NFκB activity, and/or and other pathways downstream of NOD2:RIP2 can be modulated. For example, the at least one agent can be delivered to a macrophage that is overexpressing one or more cytokines in response to MDP-induced activation of NFκB. Delivery of the at least one agent to the macrophage can prevent or inhibit formation of fully-activated NOD2:RIP2 complex formation by inhibiting Y474 RIP2 phosphorylation. In the absence of activated NOD2:RIP2 complexes, NFκB activity can be decreased or inhibited. Consequently, cytokine expression in the macrophage can be reduced or inhibited to mitigate or eliminate the inflammatory disorder in the subject.

In one example of the present application, a method is provided for treating a subject suffering from sarcoidosis. Sarcoidosis is a multisystem granulomatous inflammatory disease characterized by small inflammatory nodules (also referred to as non-caseating granulomas). Granulomatous inflammation is characterized primarily by accumulation of monocytes, macrophages and activated T-lymphocytes, with increased production of key inflammatory mediators (e.g., TNF-α, INF-γ, IL-2 and IL-12) as a result of activation of NFκB and other pathways downstream of NOD2:RIP2. To treat a subject suffering from sarcoidosis, a therapeutically effective amount of a tyrosine kinase inhibitor, such as erlotinib and/or gefitinib can be intravenously administered to the subject. Upon administration, erlotinib and/or gefitinib can contact one or more of the subject's NOD2-bearing cells (e.g., macrophages) that are overexpressing the inflammatory mediators. Delivery of erlotinib and/or gefitinib to the NOD2-bearing cell can prevent or inhibit formation of fully-activated NOD2:RIP2 complex formation by inhibiting Y474 RIP2 phosphorylation. In the absence of activated NOD2:RIP2 complexes, NFκB activity may then be decreased or inhibited. Consequently, production of inflammatory mediators by the NOD2-bearing cell can be reduced or inhibited to mitigate or eliminate sarcoidosis in the subject.

In another example of the present invention, a method is provided for treating a subject suffering from an inflammatory bowel disease, such as Crohn's disease. Crohn's disease is an inflammatory disease of the intestines that may affect any part of the gastrointestinal tract and cause a wide variety of symptoms (e.g., abdominal pain, vomiting, weight loss, skin rashes). Abnormalities in the immune system have often been invoked as being causative of Crohn's disease. There is an increasing body of evidence in favor of the hypothesis that Crohn's disease results from an impaired innate immunity, with inflammation stimulated by an over-active Tₕ1 cytokine response. Additionally, the immunodeficiency, which has been shown to be due to (at least in part) impaired cytokine secretion by macrophages, is thought to lead to a sustained microbial-induced inflammatory response (particularly in the colon where the bacterial load is especially high). To treat a subject suffering from Crohn's disease, a therapeutically effective amount of a tyrosine kinase inhibitor, such as erlotinib and/or gefitinib can be intravenously administered to the subject. Upon administration, erlotinib and/or gefitinib can contact one or more of the NOD2-bearing cells (e.g., macrophages) that are overexpressing inflammatory cytokines. Delivery of erlotinib and/or gefitinib to the NOD2-bearing cell can prevent or inhibit formation of fully-activated NOD2:RIP2 complex formation by inhibiting Y474 RIP2 phosphorylation. In the absence of activated NOD2:RIP2 complexes, NFκB activity can then be decreased or inhibited. Consequently, production of inflammatory cytokines by the NOD2-bearing cells can be reduced or inhibited to mitigate or eliminate Crohn's disease in the subject.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Inhibition of RIP2's Tyrosine Kinase Activity Limits NOD2-Driven Cytokine Responses

In this example, we found that, upon NOD2 activation, RIP2 is inducibly tyrosine-phosphorylated. We mapped the phosphorylation site to Tyr 474 (Y474) and found that phosphorylation of this tyrosine is necessary for maximal RIP2-induced NF-KB activation and maximal signaling synergy with NOD2. We further found that RIP2 possesses tyrosine kinase activity and is capable of tyrosine autophosphorylation in response to NOD2 activation. Lastly, we found that RIP2's tyrosine kinase activity can be inhibited by the epidermal growth factor receptor (EGFR) inhibitors getfitinib and erlotinib, and that inhibition can dampen NOD2-induced cytokine release and NF-κB activation in a variety of NOD2 hyperactive states. These findings suggest that inhibition of NOD2 signaling has efficacy in inflammatory diseases in which NOD2 is active. This example establishes the tyrosine phosphorylation site Y474 as a key activator and marker of activated RIP2, and it identifies pharmacologic agents already in clinical use as potential chemotherapeutics in clinical autoinflammatory disorders.

### Materials and Methods

### Cell Culture, Transfections, Immunoprecipitations, and Western Blotting

HEK293 cells were grown in DMEM (Mediatech) supplemented with 5% FBS (Hyclone) and 1% antibiotic-antimycotic (Invitrogen). RAW264.7 macrophages stably expressing NOD2 were grown in DMEM (Mediatech) supplemented with 10% FBS (Hyclone), 1% antibiotic-antimycotic (Invitrogen), and 150 μg/mL G418 (InvivoGen). HT-29 cells stably expressing NOD2 were grown in RPMI (Mediatech) supplemented with 10% FBS (Hyclone), 1% antibiotic-antimycotic (Invitrogen), and 150 μg/mL G418 (InvivoGen). BMDMs were generated by culturing bone marrow from wild-type or ITCH^{-/-} mice in Ladmac-conditioned medium for 7 d before passaging cells in DMEM with 10% FBS for downstream assays. All cell lines were obtained from American Type Tissue Collection (ATCC). Stably expressing cell lines were generated through infection with virus made by transfection of a retrovirus packaging cell line, Amphopak-293, with pMXneo-based constructs. Transient transfections were performed by calcium phosphate precipitation or by Lipofectamine LTX (RIP2^{-/-} MEFs). Lysates were obtained using Cell Signaling lysis buffer (50 mM Tris at pH 7.5, 150 mM NaCl, 1% Triton X-100, 1 mM EDTA, 1 mM EGTA, 1 mM β-glycerophosphate, 1 mM PMSF, 1 mM NaV04, 10 nM Calyculin A, protease inhibitor cocktail [Sigma]). After adding protein G sepharose beads, immunoprecipitations were washed at least five times before Western blotting. Western blotting was performed on nitrocellulose membranes (Bio-Rad) or on PVDF membranes (Whatmann) as described previously (Abbott et al. 2004).

### Antibodies, Plasmids, and Reagents

Anti-phosphotyrosine (P-Tyr-100), anti-phospho IKBa (5A5), anti-total IκBα (44D4), anti-phospho EGFR (53A5), and anti-EGFR Alexa 488 (D38B1) were obtained from Cell Signaling Technology. Anti-Omni rabbit (M-21), anti-Omni mouse (D-8), anti-RICK (H-300), and anti-ITCH (H-100) were obtained from Santa Cruz Biotechnology. Anti-HA (16B12) was obtained from Covance. Streptavidin agarose was obtained from Sigma, while protein G agarose was obtained from Invitrogen. The K47A, T95M, Y474F, Y520, and Y520F/Y474F RIP2 constructs were generated by QuickChange site-directed mutagenesis of the Omni-RIP2 construct (Stratagene) and were all sequence-verified. Likewise, the R334Q NOD2 construct was also generated by site-directed mutagenesis of pMXneo-Flag-Nod2. HA-Ubiquitin, Omni-RIP2, NTAP-RIP2, Omni-NOD2, Flag-NOD2, Flag-L1007insC NOD2, Flag-ITCH, and Flag-C830A ITCH were used. TheOmni-tagged NOD2 construct was originally subcloned from the mouse EST. Unlike the human, the insertion of cytosine at L1007 in the mouse allele causes a frameshift, leading to a nonsense transcript encoding 41 additional base pairs. HA-NOD2 and HA-NOD2 deletion mutants (ΔCARDs, ΔLRRs, and CARDs) were a kind gift from Christine McDonald (Cleveland Clinic Foundation). MDP was obtained from Bachem, and LPS was obtained in a highly purified form from InvivoGen. Gefitinib and erlotinib were obtained from LC Laboratories.

### Mass Spectrometry

HT-29 cells (1×10⁹) were treated with 10 μg/mL MDP prior to lysis in Cell Signaling lysis buffer (recipe above) containing pervanadate. Endogenous RIP2 was immunoprecipitated using an anti-RIP2 antibody (H300; Santa Cruz Biotechnology) with stringent washing (four washes with lysis buffer, four washes with lysis buffer containing 1 M NaCl and 1% SDS, four washes with PBS, and two washes with lysis buffer). SDS-PAGE was performed, and the predominant band (as stained by Coomassie G) was excised from the gel reduced with DTT, alkylated with iodoacetamide, and digested overnight with trypsin. Tryptic peptides were analyzed by data-dependent reversed-phase micro-capillary tandem MS (LC/MS/MS) via CID using a hybrid linear ion trap-LTQ Orbitrap XL mass spectrometer (Thermo Scientific) operated in positive ion mode at a flow rate of 300 mL/min using a 75-μm (ID)×15-μm (ID tip)×15-cm (length) C₁₈ microcapillary column. The column was equilibrated and peptides were loaded using buffer A (0.1% formic acid, 0.9% acetonitrile, 99% water) then eluted with a gradient from 3% buffer B (acetonitrile) to 38% B. One MS survey scan was followed by six MS/MS scans. The Sequest algorithm (Thermo Scientific) was used for database searching of all MS/MS spectra against the reversed SWISS-PROT protein database and single entry RIP2 database with the differential modifications: oxidation (+15.99 Da) of Met and phosphorylation (+79.97 Da) of Ser/Thr/Tyr. Peptide sequences were accepted if they matched the forward database and passed the following scoring thresholds: 2⁺ ions, Xcorr≧2.0, Sf≧0.4, P≧0; 3⁺ ions, Xcorr≧2.65, Sf≧0.5, P≧5. Peptides with gas phase charges of 1⁺ and 4⁺ were generally not accepted as valid due to difficulty of interpretation. After passing the scoring thresholds, all MS/MS were then manually inspected rigorously to be sure that all b⁻ (fragment ions resulting from the peptide's N terminus) and y⁻ ions (fragment ions resulting from the peptide's C terminus) aligned with the assigned protein database sequence. Peptide false discovery rates (FDR) were calculated to be ˜1.5% based on reversed database hits.

### In Vitro Kinase Assay

Wild-type RIP2 or K47A RIP2 was immunoprecipitated via its Omni-tag as described above. The kinase buffer contained 50 mM Tris (pH 7.5), 10 mM MgCl₂, 1 mM β-glycerophosphate, 1 mM DTT, 1 mM NaVO4, and 100 μM ATP/reaction mix. Reaction mixes were incubated for 30 min at 30° C. ³²γATP was added to the reaction.

siRNA Transfections

Four separate siRNAs against ITCH were purchased from Qiagen. The sequences of these were as follows: siRNA1, CACGGGCGAGTTTACTATGTA (SEQ ID NO: 1); siRNA2, CAAGAGCTATGAGCAACTGAA (SEQ ID NO: 2); siRNA3, ATGGGTAGCCTCACCATGAAA (SEQ ID NO: 3); siRNA4, TGCCGCCGACAAATACAAATA (SEQ ID NO: 4). The siRNA targeting the 39UTR or endogenous RIP2 had the following sequence: AAGAAGAAATGTGTTTCATAA (SEQ ID NO: 5). Six-centimeter plates of HEK293 cells were transfected with 2 nM each siRNA using calcium phosphate.

### NF-κB Dual Luciferase Assay

The dual luciferase reporter assay system was purchased from Promega and used according to the manufacturer's instructions. HEK293 cells were transfected with 100 ng of NF-κB-firefly luciferase and 50 ng of CMV-renilla luciferase along with 3 μg of the construct of interest. For RIP2^{-/-} MEFs, 2 μg of total DNA was transfected using Lipofectamine LTX (Invitrogen). Lysates were harvested 24 h after transfection using 1× passive lysis buffer (Promega). Twenty microliters of lysate was used for each assay. A Victor Plate Reader (Perkin Elmer) was used for detection of luminescence.

### Quantitative RT-PCR

RNA was harvested using a Qiagen Total RNA Isolation kit. First-strand DNA synthesis was performed using Qiagen's Quantitect Reverse Transcription kit according to the manufacturer's instructions. Real-time PCR was carried out using primers against human IL-6 (forward, 5′-TCCACAAGCGCCTTCGGTCC-3′) (SEQ ID NO: 6); reverse, 5′-GTGGCTGTCTGTGTGGGGCG-3′) (SEQ ID NO: 7) and human GAPDH (forward, 5′-GACCTGACCTGCCGTCTA-3′(SEQ ID NO: 8); re-verse, 5′-GTTGCTGTAGCCAAATTCGTT-3′) (SEQ ID NO: 9) along with the iQ SYBR Green Supermix (Bio-Rad), and was detected using a Bio-Rad iCycler. The data shown are normalized to GAPDH.

### ELISA

A mouse TNF-α ELISA kit was purchased from eBioscience and used according to the manufacturer's instructions. Supernatants from stimulated BMDM were harvested 24 h after agonist stimulation and plated overnight on ELISA plates coated with TNF-α capture antibody. Development was performed as suggested by the manufacturer. A Victor Plate Reader (Perkin Elmer) was used for detection of fluorescence.

### Flow Cytometry

One-million cells from each cell line were fixed and permeabilized using Fix/Perm and permabilization buffer reagents (eBio-science). One microgram of anti-EGFR Alexa 488 antibody (Cell Signaling) was used to stain for intracellular EGFR. After 1 h of staining, cells were thoroughly washed with permeabilization buffer and resuspended in flow cytometry buffer before acquisition of data on a BD FACscan. Data were analyzed using FlowJo software.

### Results

### RIP2 is Tyrosine-Phosphorylated in Response to NOD2 Activation

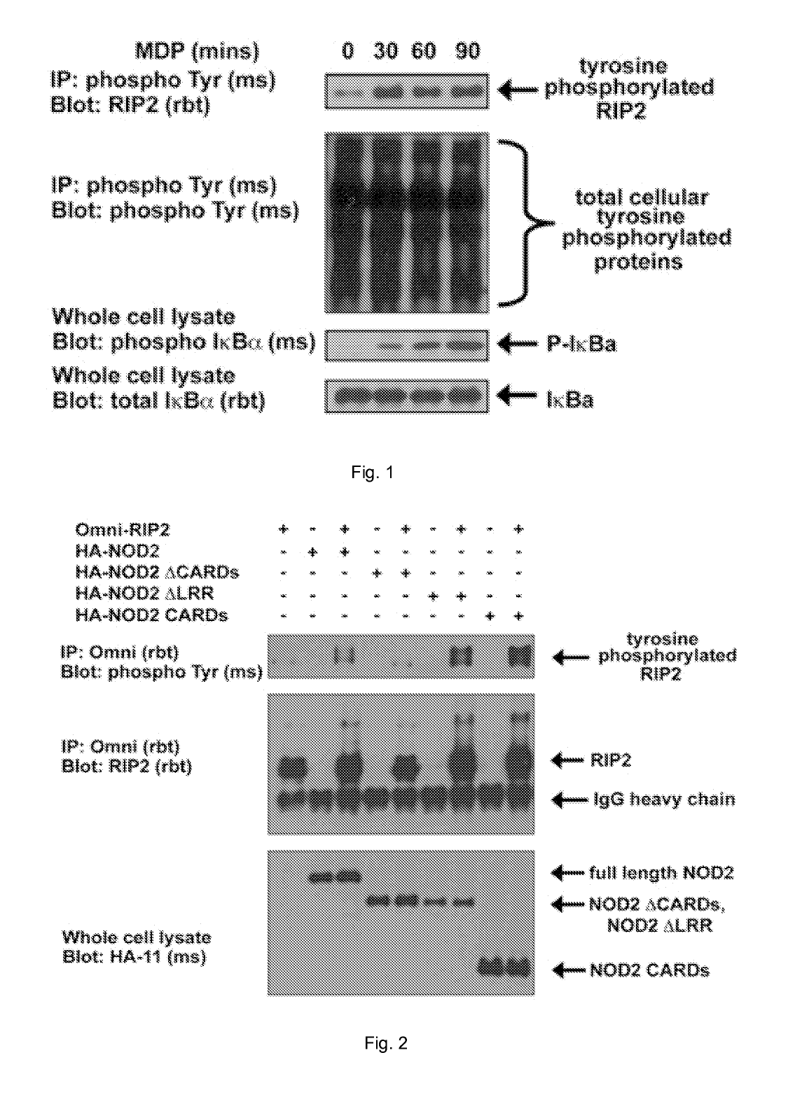

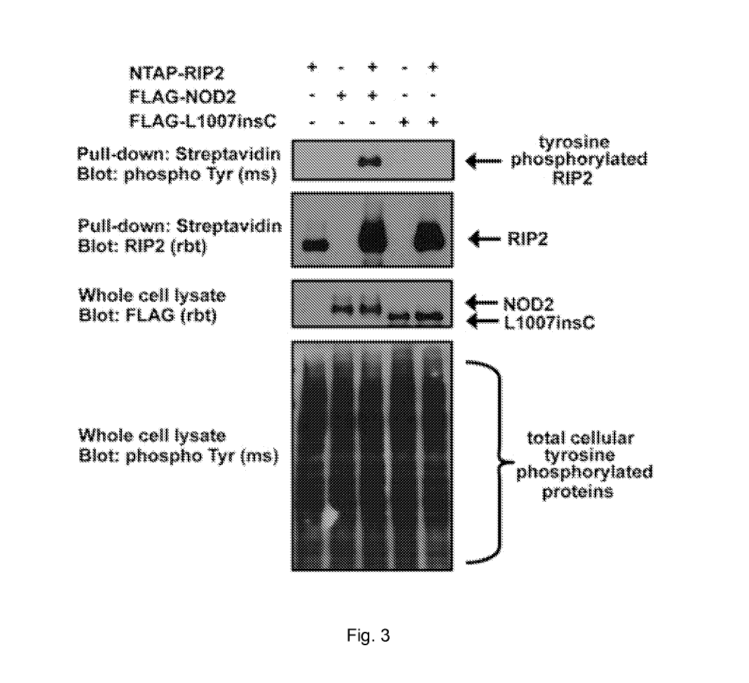

We treated human intestinal epithelial cells (HT-29) that stably express NOD2 with the NOD2 agonist MDP (a breakdown product of bacterial peptidoglycan). We found that RIP2 could be tyrosine-phosphorylated in response to MDP, and the kinetics of this MDP-induced tyrosine phosphorylation coincided with activation of the NF-kB pathway (as shown by the appearance of phospho-IkBa) (FIG. 1). Transient transfection experiments also showed that overexpression of NOD2 can itself cause RIP2 tyrosine phosphorylation (FIG. 2). This RIP2 modification is dependent on the NOD2 CARDs. These CARDs are important for mediating the NOD2:RIP2 interaction, and use of a NOD2 mutant lacking this region (ACARDs) abolishes the NOD2-induced RIP2 tyrosine phosphorylation (FIG. 2). Consistent with these results, transient transfections of a NOD2 Crohn's disease-associated polymorphism, L1007insC NOD2, failed to induce RIP2 tyrosine phosphorylation (FIG. 3). The L1007insC allele has been found by multiple groups to bind RIP2 less efficiently than its wild-type counterpart, and this finding again suggests that NOD2 binding is required for RIP2 tyrosine phosphorylation. Collectively, these findings show that activation of NOD2 (through exposure to MDP or overexpression of NOD2) and a direct interaction between NOD2 and RIP2 are required for tyrosine phosphorylation of RIP2.

### RIP2 is Phosphorylated on Tyr 474

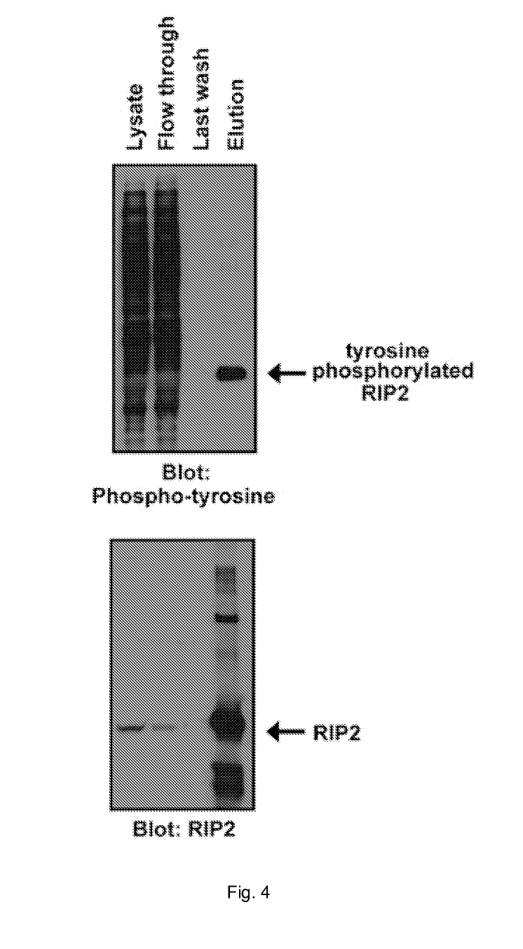

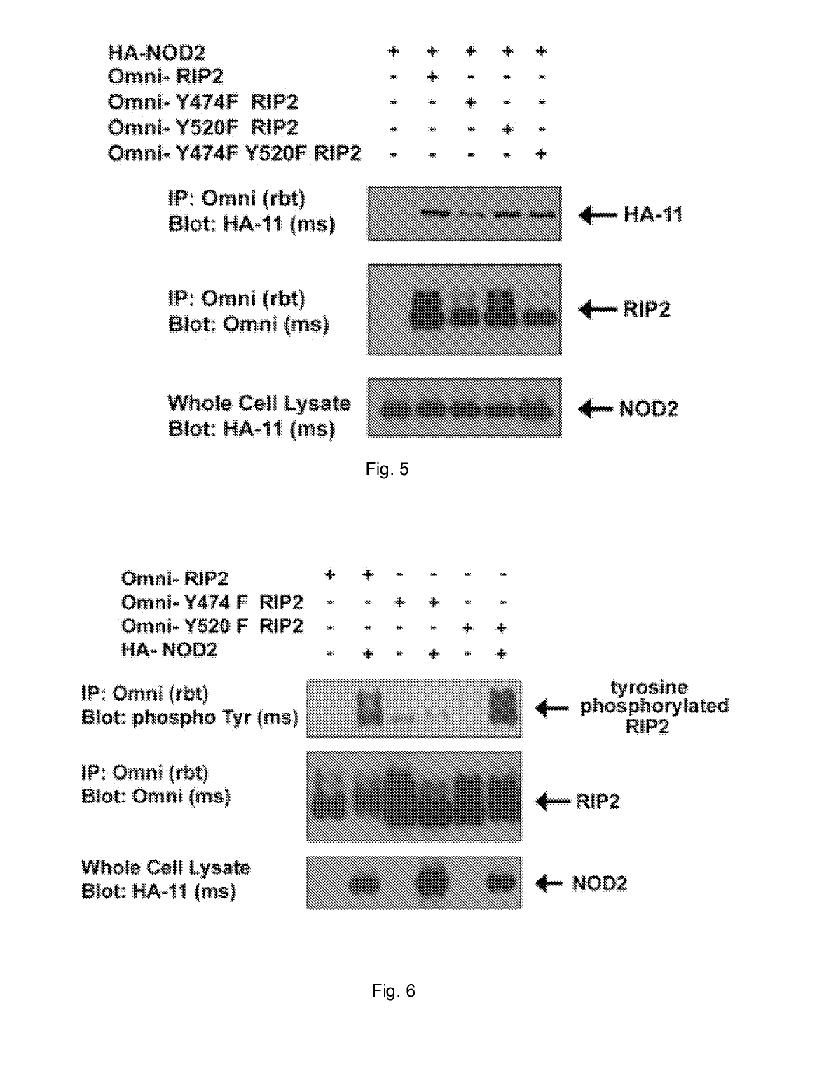

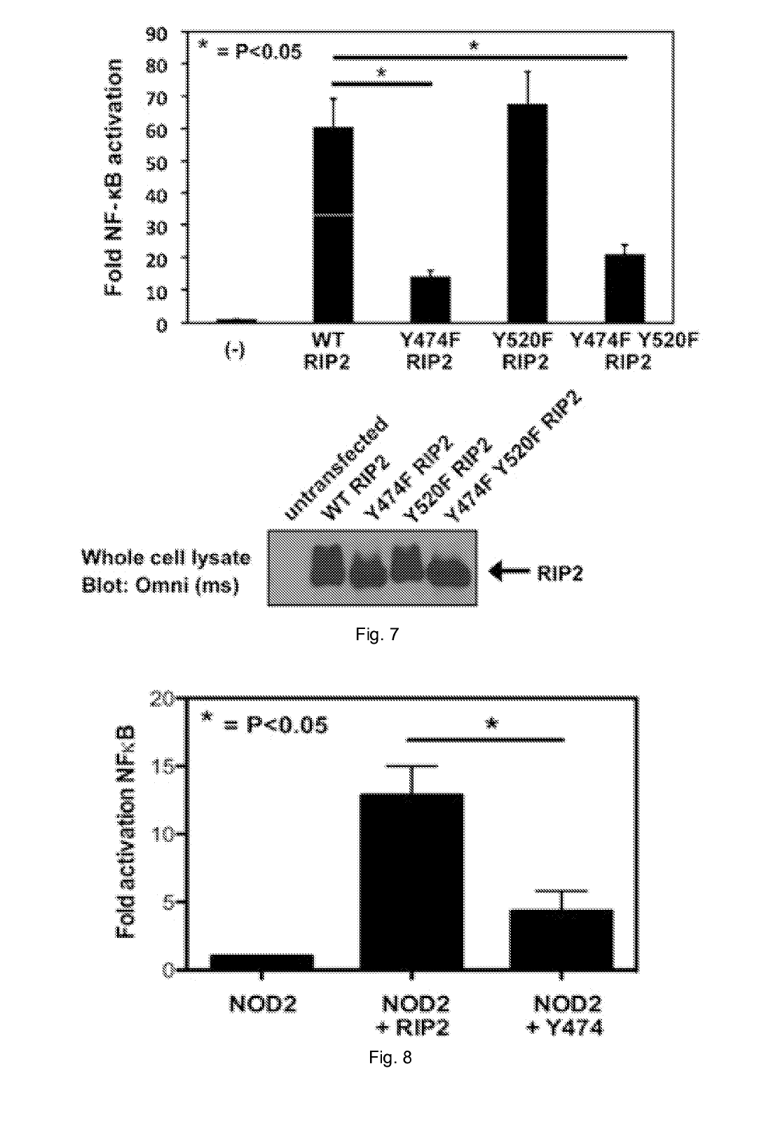

To determine the site at which the MDP/NOD2-induced RIP2 tyrosine phosphorylation occurs, HT-29 cells that express endogenous RIP2 were exposed to MDP for 45 min. Lysates were harvested, and endogenous RIP2 was immunoprecipitated under stringent conditions (1% SDS, 1 M NaCl). While the whole lysate contained a multitude of tyrosine-phosphorylated proteins, a single tyrosine-phosphorylated protein was identified in the RIP2 immunoprecipitate (FIG. 4). LC/MS/MS analyses showed that this band contained almost exclusively peptides from RIP2 (74 total RIP2 peptides) and contained phosphorylation of endogenous RIP2 at Tyr 474 (Y474) (FIG. 4). Additional experiments using transfected 293s were also performed and resulted in the identification of two additional tyrosine phosphorylation sites (Y381 and Y520). These sets of experiments, coupled with the findings from published post-translational modification databases, ultimately resulted in the identification of three tyrosine phosphorylation sites (Y381, Y474, and Y527) on RIP2. As only Y474 and Y527 are conserved in zebrafish (the earliest organism in which RIP2 is expressed), we focused on these two sites. In order to confirm the identity of the MDP/NOD2-induced RIP2 tyrosine phosphorylation site, we performed site-directed mutagenesis on Y474 and Y520 and mutated each of the tyrosines to phenylalanine (Y474F and Y520F). Y474F RIP2, Y520F RIP2, and the double mutant Y474FY520F RIP2 all bind to NOD2; however, we consistently find slightly decreased binding of Y474F to NOD2 (FIG. 5), and transient transfection assays show that mutation on Y474 but not Y520 abolished NOD2-induced RIP2 tyrosine phosphorylation (FIG. 6). To then determine whether downstream NF-kB activation is affected by phosphorylation of RIP2 at Y474, we transfected HEK293 cells with NF-KB-driven luciferase, CMV-driven renilla, and each of the following: wild-type RIP2, Y474F RIP2, Y520F RIP2, or Y474FY520F RIP2. Prevention of phosphorylation at Y474 reduced (but did not abolish) NF-κB activation significantly compared with the activation induced by wild-type RIP2 or the activation induced by Y520F RIP2 (FIG. 7). In addition, RIP2^{-/-} mouse embryonic fibroblasts (MEFs) showed a severely decreased NF-KB response when NOD2 was cotransfected into the cell in the presence of Y474F RIP2, but not when cotransfected into the cell in the presence of wild-type RIP2. Lastly, in a third set of experiments, endogenous RIP2 expression was inhibited by using a siRNA that targets RIP2's 39 untranslated region (UTR). While this siRNA was about 80% to about 90% effective at inhibiting endogenous RIP2 expression, the wild-type RIP2 and Y474F RIP2 constructs that did not have this 3′UTR were unaffected by this siRNA (FIG. 8). Use of the endogenously targeting RIP2 siRNA showed that the synergy between NOD2 and RIP2 was significantly decreased when Y474F RIP2 was transfected into the cell (FIG. 9). Lastly, collectively, these data show that Y474 is a functional tyrosine phosphorylation site, as Y474 phosphorylation is induced by NOD2 activation and is necessary for maximal NOD2-driven NF-KB activation.

### RIP2 Autophosphorylates on Tyrosines in Response to NOD2 Activation

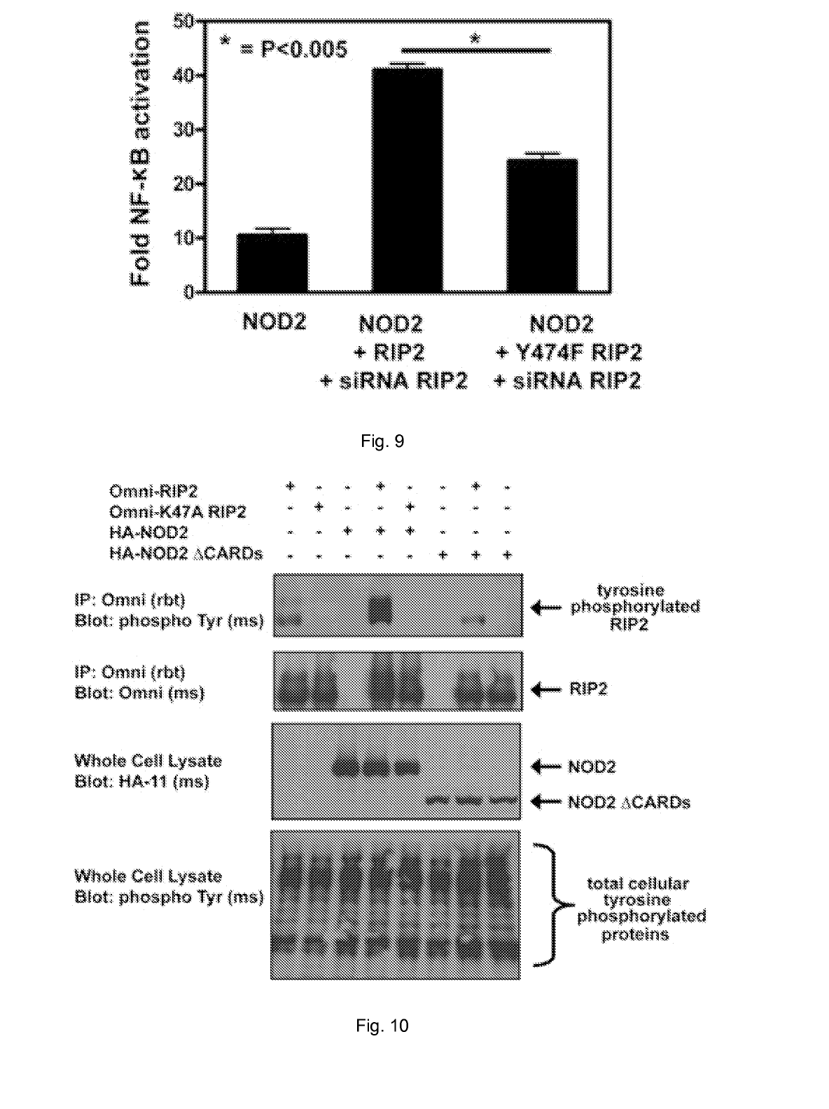

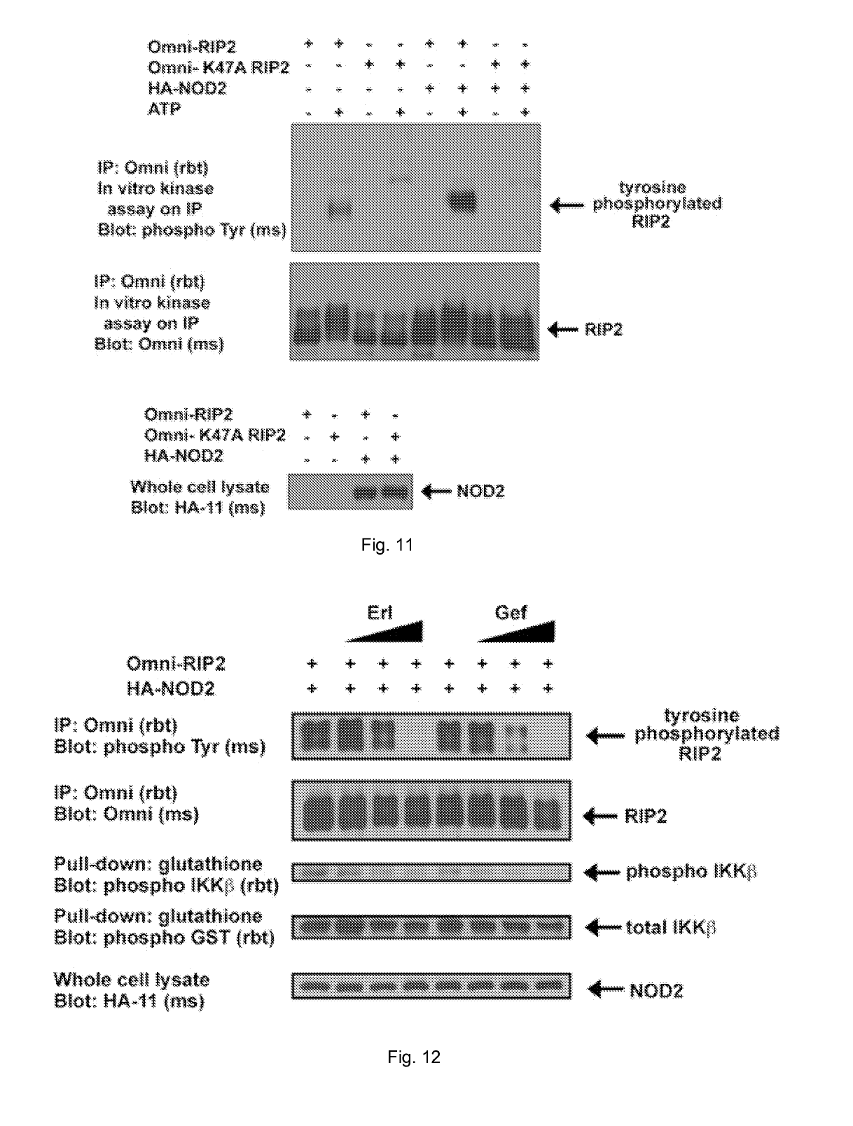

We tested the hypothesis that RIP2 might be capable of tyrosine auto-phosphorylation. To address this, we used a kinase-inactive form of RIP2 (K47A). Transient transfection assays showed that wild-type but not K47A RIP2 is tyrosine-phosphorylated in response to overexpression of NOD2 F4 (FIG. 10). As a control, this inducible tyrosine phosphorylation is also lost if we cotransfect NOD2 lacking the CARD regions (and which is therefore unable to recruit and activate RIP2) with either the wild-type or kinase-inactive RIP2 (FIG. 10). To demonstrate in vitro tyrosine kinase autophosphorylation activity, we performed in vitro kinase assays using RIP2 as the substrate. After transient transfection with NOD2 and either wild-type or kinase-inactive RIP2, RIP2 was stringently immunoprecipitated, and this immunoprecipitate was incubated in the presence or absence of ATP in a standard in vitro kinase assay. While wild-type RIP2 was capable of autophosphorylation on tyrosine, kinase-inactive (K47A) RIP2 was unable to induce autophosphorylation on tyrosine either basally or upon cotransfection with NOD2 (FIG. 11). Collectively, these data show that it is, in fact, the intrinsic tyrosine kinase activity of RIP2 that is responsible for MDP/NOD2-induced RIP2 tyrosine phosphorylation.

### Erlotinib and Gefitinib Inhibit RIP2 Tyrosine Phosphorylation

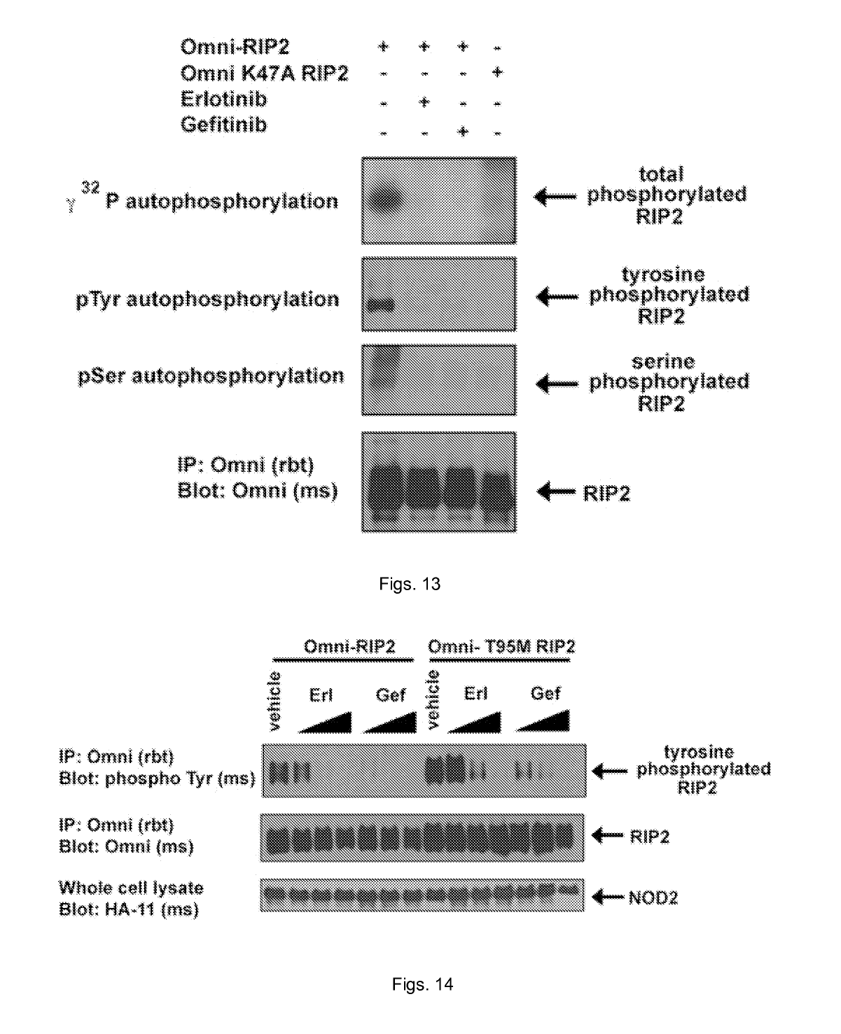

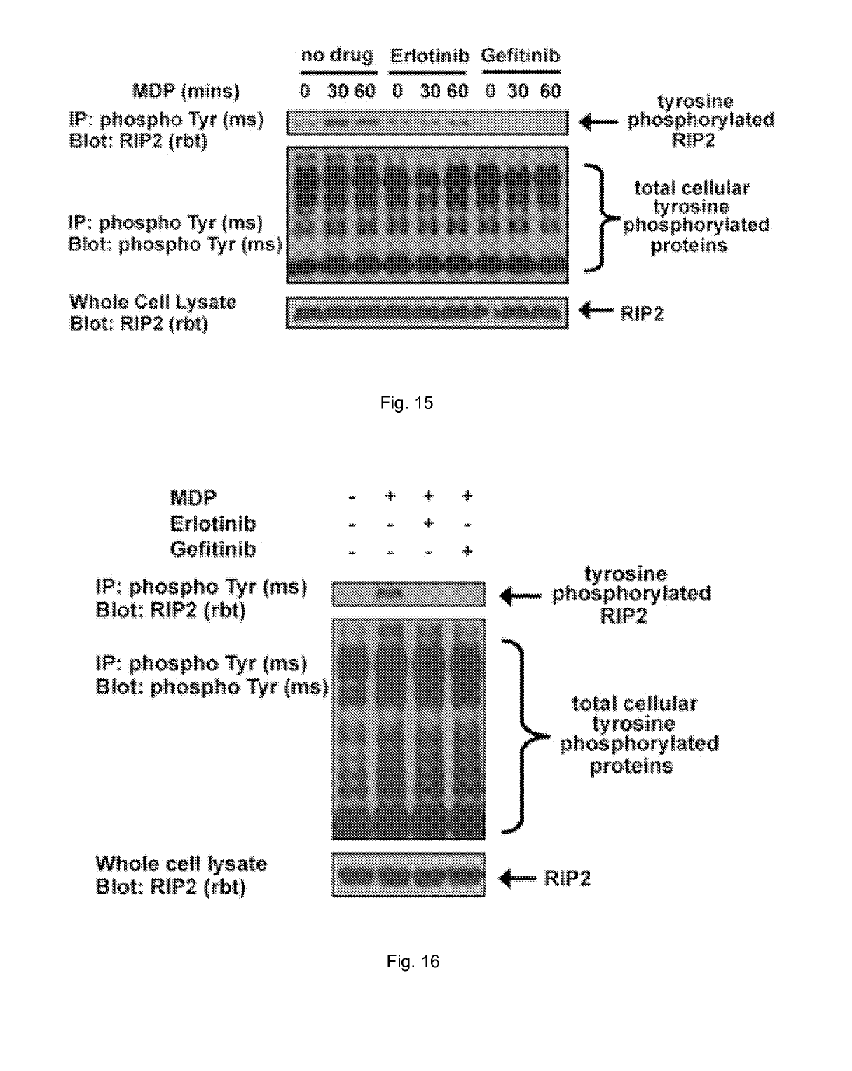

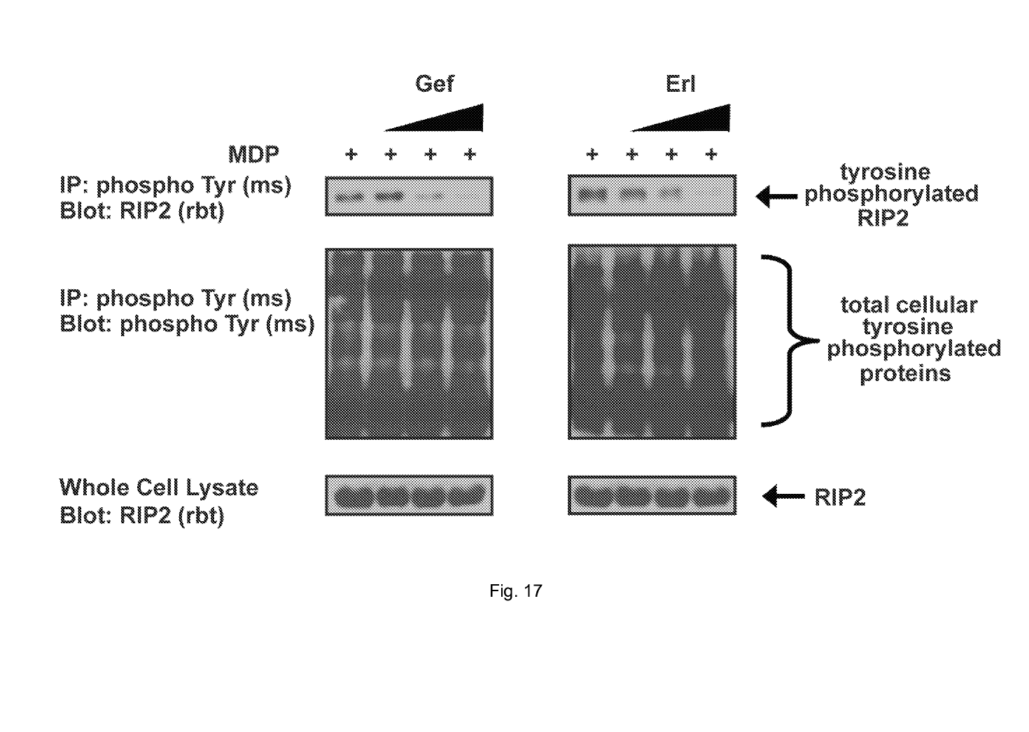

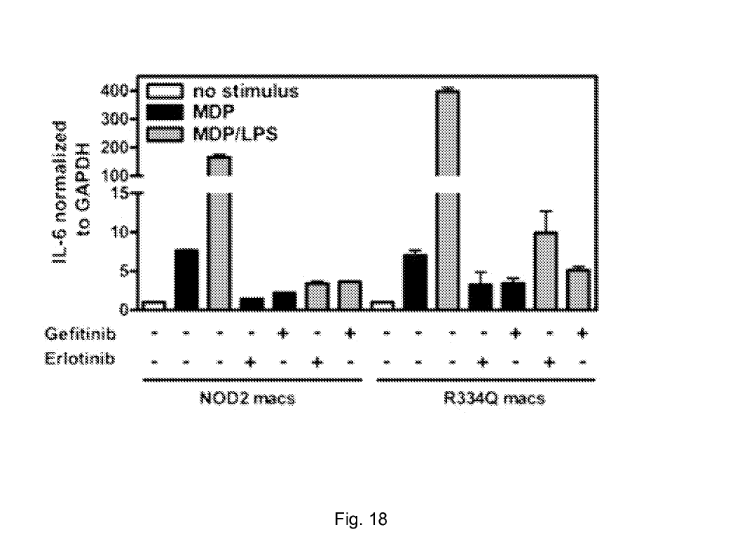

Our findings show that RIP2 autophosphorylates on tyrosine in response toNOD2 activation and that such tyrosine phosphorylation correlates with NF-κB activity. Given that NOD2:RIP2 inhibition might be efficacious in hyperactive NOD2 signaling state (e.g., in individuals with Blau Syndrome and Early Onset Sarcoidosis who harbor an activating NOD2 mutation, in wild-type NOD2 pediatric Crohn's disease patients who are reported to have increased RIP2 kinase activity, and in patients harboring ITCH mutations who exhibit increased mucosal inflammatory pathology), we performed a limited small-molecule inhibitor screen to identify RIP2 tyrosine kinase inhibitors. Three compounds were found to strongly inhibit the tyrosine kinase activity of RIP2. These compounds included SB203580 (a p38 inhibitor), gefitinib (an EGFR inhibitor), and erlotinib (an EGFR inhibitor). Erlotinib and gefitinib were found to inhibit tyrosine phosphorylation of RIP2 and RIP2-induced IKKβ activation with similar efficacy (FIG. 12). In vitro kinase assays indicated that these drugs could inhibit both tyrosine and serine-threonine kinase activity of RIP2, as shown by both Western blot and loss of total ³²P incorporation in radio-active autophosphorylation assays (FIG. 13). While these drugs act as competitive inhibitors for ATP by binding to the ATP-binding pocket of their target kinase, neither gefitinib (Iressa) nor erlotinib (Tarceva) has been shown previously to have an effect on NOD2:RIP2 signaling or MDP-induced cytokine release. For this reason, we next tested the specificity of these agents for RIP2. As shown in FIG. 14, the inhibition of RIP2 tyrosine phosphorylation by both erlotinib and gefitinib occurs at nanomolar concentrations (i.e., at 100 nM, 500 nM, and 2 μM). These doses are similar to the doses shown to have clinical efficacy when targeting the EGFR. Patients who develop resistance to erlotinib and gefitinib harbor a mutation in the ATP-binding pocket of EGFR (T790M) that prevents binding of the drug and subsequently causes insensitivity to these compounds. In order to determine whether these EGFR inhibitors act on RIP2 directly, we generated RIP2 containing the homologous desensitizing mutation in the ATP-binding pocket (T95M). This mutation partially desensitized RIP2 to the inhibitory effects of erlotinib and gefitinib (FIG. 14), suggesting that the effect of erlotinib and gefitinib on RIP2 was direct. In addition, we also show that these drugs can inhibit endogenous MDP-induced RIP2 tyrosine phosphorylation in both an intestinal epithelial cell line and a macrophage cell line, both of which stably express NOD2 (FIGS. 15, 16). While these drugs have no effect on the NOD2:RIP2 interaction, they can inhibit endogenous RIP2 tyrosine phosphorylation in a dose-dependent manner (FIG. 17). Lastly, to mimic a hyperactive NOD2 state, we studied the effect of erlotinib and gefitinib on cells expressing activating Blau Syndrome-causing NOD2 mutations. RAW264.7 cells were stably transduced with either wild-type NOD2 or NOD2 harboring the gain-of-function Blau Syndrome-causing mutation R334Q. Cells were then treated with MDP alone or LPS (lipopolysaccharide)+MDP to study the well-described NOD2/TLR (Toll-like receptor) synergy. If RIP2 tyrosine phosphorylation is a necessary precursor to NF-κB activation, then inhibition of RIP2 tyrosine phosphorylation using erlotinib and gefitinib may serve to correct the excessive activation seen with the R334Q polymorphism. Under stimulatory conditions, both erlotinib and gefitinib reduced the levels of IL-6 (interleukin-6) in response to MDP or synergistic NOD2-TLR stimulation, suggesting that the use of such drugs is efficacious (FIG. 18).

### Example 2

Following the work performed in Example 1, we have been moving the work into in vivo disease models. Given the relative ease of administration via inhalation or via intratracheal administration of agents into mice, the relative ease of obtaining bronchoalveolar lavage (BAL) specimens from both mice and patients and the fact that the NOD2 pathway seems to be hyperactivated in a number of inflammatory lung diseases (sarcoidosis and asthma in particular), we have been focusing on sarcoidosis with the goal of additionally studying asthma models. To this end, we have mastered a model of sarcoidosis. In this model, heat-killed P. acnes (a bacteria frequently per'd from granulomas of sarcoid patients) is injected intraperitoneally into mice. Two weeks later, heat-killed P. acnes is injected intratracheally into these same mice. 9 days after this, BALs are performed and the lungs are harvested. Histologically, there is perivascular and peribronchiolar inflammation as well as whorled lesions resembling granulomas and macrophages within the alveolar space. While this has been published as a model of sarcoidosis, there is considerable overlap with asthma in regards to the histopathology seen in this model of disease when compared to that seen in asthma.

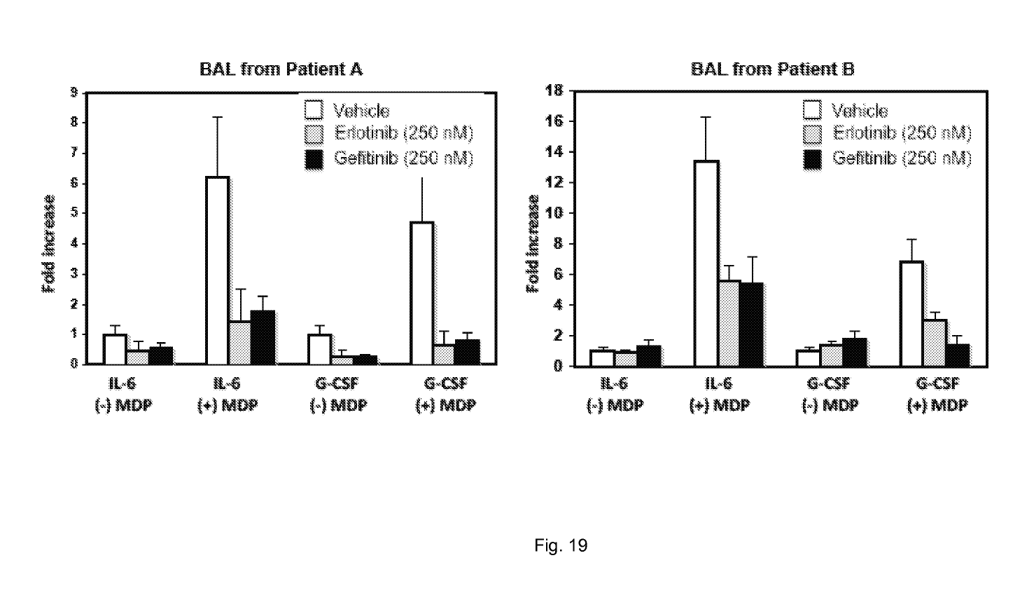

If we carry this experiment a step further and utilize ITCH^{-/-} mice, we see an exacerbated response relative to WT mice. ITCH^{-/-} mice show an increased MDP-driven NOD2:RIP2 signal activation and show increased NOD2:RIP2-dependent cytokine release. This situation mimics that seen in Early Onset Sarcoidosis (with genetic gain-of-function NOD2 variants) or, by extension, asthma (with NOD2 activation causing decreased airway tolerance). In this set of experiments, the amount of P. acnes used was decreased to enhance the signal to noise ratio. With this decreased amount of P. acnes used, the BALs from the ITCH^{-/-} mice showed a dramatic elevation in the numbers of neutrophils present relative to WT mice, and at these doses, the histologic response was much more severe in the ITCH^{-/-} mice (not shown but available upon request). Notably, this response occurred regardless of whether the mouse was pre-injected with heatkilled P. acnes 2 weeks prior to intratracheal inoculation versus simply intratracheally inoculated with heat-killed P. ances with no prior intraperitoneal injection. This finding suggests that a faulty innate immune system is at work, supporting the hypothesis that aberrant NOD2:RIP2 activation enhances disease. Lastly, we have extended this work to human patients. We have obtained BALs from sarcoidosis patients and we have shown that the exaggerated MDP-induced cytokine release from these patients is attenuated with the NOD2:RIP2 signaling pathway is inhibited through the use of nanomolar concentrations of gefitinib or erlotinib (FIG. 19).

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1-8. (canceled)

9. A method for treating an inflammatory disorder and/or immunological disorder associated with MDP-induced, NFκB activation in a subject, the method comprising:
administering to the subject a therapeutically effective amount of at least one EGFR inhibitor; wherein the at least one EGFR inhibitor inhibits RIP2 kinase activity and phosphorylation of the NOD2:RIP2 complex in a NOD2-bearing cell of the subject.

10. The method of claim 9, the inflammatory disease being selected from the group consisting of sacroidosis, rheumatoid arthritis, Crohn's disease, Blau syndrome, early onset sarcoidosis, colitis, asthma, graft versus host disease, and inflammatory bowel disease.

11. The method of claim 9, the EGFR inhibitor inhibiting phosphorylation of Y474 RIP2 tyrosine of a NOD2:RIP signaling complex.

12. The method of claim 9, the EGFR inhibitor comprising a quinazoline derivative having the following general formula: 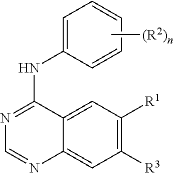
wherein
n is 1, 2 or 3 and each R² is independently halogeno, trifluoromethyl or (1-4C)alkyl;
R³ is (1-4C)alkoxy; and
R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy,
and wherein any of the above-mentioned R¹ substituents comprising a CH₂ (methylene) group which is not attached to a N or O atom optionally bears on said CH₂ group a hydroxy substituent;
or a pharmaceutically-acceptable salt thereof.

13. The method of claim 9, the EGFR inhibitor comprising a quinazoline derivative having the following general formula: 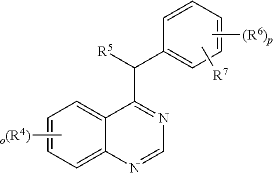
and to pharmaceutically acceptable salts and prodrugs thereof,
wherein:
o is 1, 2, or 3;
each R⁴ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R⁴ is independently selected from R¹² and (C₁-C₄)-alkyl substituted by cyano, wherein R¹² is selected from the group consisting of R⁸, —OR⁹, —NR⁹ R⁹, —C(O)R¹⁰, —NHOR⁸, —OC(O)R⁹, cyano, A and —YR⁸; R⁸ is C₁-C₄ alkyl; R⁹ is independently hydrogen or R⁸; R¹⁰ is OR⁹ or —NR⁹ R⁹; A is selected from piperidino, morpholino, pyrrolidino, 4-R⁹-piperazin-1-yl, imidazol-1-yl, 4-pyridon-1-yl, —(C₁-C₄ alkylene)(CO₂ H), phenoxy, phenyl, phenylsulfanyl, C₂-C₄ alkenyl, and —(C₁-C₄ alkylene)C(O)NR⁹ R⁹; and Y is S, SO, or SO₂; wherein the alkyl moieties in R⁸, —OR⁹ and —NR⁹ R⁹ are optionally substituted by one to three substituents independently selected from halo and R¹², and wherein the alkyl moieties of said optional substituents are optionally substituted by halo or R¹², with the proviso that two heteroatoms are not attached to the same carbon atom, and with the further proviso that no more than three R¹² groups may comprise a single R⁴ group;
or each R⁴ is independently selected from—NHSO₂ R⁸, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹³—(C₂-C₄)-alkanoylamino wherein R¹³ is selected from halo, —OR⁹, C₂-C₄ alkanoyloxy, —C(O)R¹⁰, and —NR⁹ R⁹; and wherein the foregoing R⁴ groups are optionally substituted by 1 or 2 substituents independently selected from halo, C₁-C₄ alkyl, cyano, methanesulfonyl and C₁-C₄ alkoxy;
or two R⁴ groups are taken together with the carbons to which they are attached to form a 5-8 membered ring that includes 1 or 2 heteroatoms selected from O, S and N;
R⁵ is hydrogen or C₁-C₆ alkyl optionally substituted by 1 to 3 substituents independently selected from halo, C₁-C₄ alkoxy, —NR⁹ R⁹, and —SO₂ R⁸;
p is 1 or 2 and each R³ is independently selected from hydrogen, halo, hydroxy, C₁-C₆ alkyl, —NR⁹ R⁹, and C₁-C₄ alkoxy, wherein the alkyl moieties of said R⁶ groups are optionally substituted by 1 to 3 substituents independently selected from halo, C₁-C₄ alkoxy, —NR⁹ R⁹, and —SO₂ R⁸; and,
R⁷ is azido or -(ethynyl)-R¹⁴ wherein R¹⁴ is hydrogen or C₁-C₆ alkyl optionally substituted by hydroxy, —OR⁹, or —NR⁹ R⁹.

14. The method of claim 9, wherein the EGFR inhibitor comprises at least one of erlotinib or gefitinib.

15-28. (canceled)

29. A method for treating sarcoidosis in a subject, the method comprising:
administering to the subject a therapeutically effective amount of at least one tyrosine kinase inhibitor that inhibits nucleotide-binding oligomerization domain containing 2 (NOD2):receptor-interacting protein 2 (RIP2) signaling in a NOD2-bearing cell, wherein the amount is not cytotoxic to the cell, wherein the tyrosine kinase inhibitor comprises an epidermal growth factor receptor (EGFR) inhibitor.

30. The method of claim 29, the disease being associated with muramyl dipeptide (MDP)-induced, NFκB activation.

31. The method of claim 29, the tyrosine kinase inhibitor inhibiting phosphorylation of Y474 RIP2 tyrosine of a NOD2:RIP signaling complex.

32. (canceled)

33. The method of claim 29, the EGFR inhibitor comprising a quinazoline derivative having the following general formula: 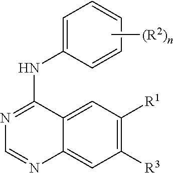
wherein
n is 1, 2 or 3 and each R² is independently halogeno, trifluoromethyl or (1-4C)alkyl;
R³ is (1-4C)alkoxy; and
R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy,
and wherein any of the above-mentioned R¹ substituents comprising a CH₂ (methylene) group which is not attached to a N or O atom optionally bears on said CH₂ group a hydroxy substituent;
or a pharmaceutically-acceptable salt thereof.

34. The method of claim 29, the EGFR inhibitor comprising a quinazoline derivative having the following general formula: 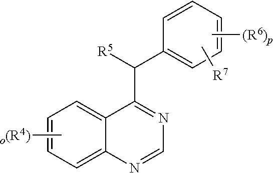
and to pharmaceutically acceptable salts and prodrugs thereof,
wherein:
o is 1, 2, or 3;
each R⁴ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R⁴ is independently selected from R¹² and (C₁-C₄)-alkyl substituted by cyano, wherein R¹² is selected from the group consisting of R⁸, —OR⁹, —NR⁹ R⁹, —C(O)R¹⁰, —NHOR⁸, —OC(O)R⁹, cyano, A and —YR⁸; R⁸ is C₁-C₄ alkyl; R⁹ is independently hydrogen or R⁸; R¹⁰ is —OR⁹ or —NR⁹ R⁹; A is selected from piperidino, morpholino, pyrrolidino, 4-R⁹-piperazin-1-yl, imidazol-1-yl, 4-pyridon-1-yl, —(C₁-C₄ alkylene)(CO₂ H), phenoxy, phenyl, phenylsulfanyl, C₂-C₄ alkenyl, and —(C₁-C₄ alkylene)C(O)NR⁹ R⁹; and Y is S, SO, or SO₂; wherein the alkyl moieties in R⁸, —OR⁹ and —NR⁹ R⁹ are optionally substituted by one to three substituents independently selected from halo and R¹², and wherein the alkyl moieties of said optional substituents are optionally substituted by halo or R¹², with the proviso that two heteroatoms are not attached to the same carbon atom, and with the further proviso that no more than three R¹² groups may comprise a single R⁴ group;
or each R⁴ is independently selected from —NHSO₂ R⁸, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹³—(C₂-C₄)-alkanoylamino wherein R¹³ is selected from halo, —OR⁹, C₂-C₄ alkanoyloxy, —C(O)R¹⁰, and —NR⁹ R⁹; and wherein the foregoing R⁴ groups are optionally substituted by 1 or 2 substituents independently selected from halo, C₁-C₄ alkyl, cyano, methanesulfonyl and C₁-C₄ alkoxy;
or two R⁴ groups are taken together with the carbons to which they are attached to form a 5-8 membered ring that includes 1 or 2 heteroatoms selected from O, S and N;
R⁵ is hydrogen or C₁-C₆ alkyl optionally substituted by 1 to 3 substituents independently selected from halo, C₁-C₄ alkoxy, —NR⁹ R⁹, and —SO₂ R⁸;
p is 1 or 2 and each R³ is independently selected from hydrogen, halo, hydroxy, C₁-C₆ alkyl, —NR⁹ R⁹, and C₁-C₄ alkoxy, wherein the alkyl moieties of said R⁶ groups are optionally substituted by 1 to 3 substituents independently selected from halo, C₁-C₄ alkoxy, —NR⁹ R⁹, and —SO₂ R⁸; and,
R⁷ is azido or -(ethynyl)-R¹⁴ wherein R¹⁴ is hydrogen or C₁-C₆ alkyl optionally substituted by hydroxy, —OR⁹, or —NR⁹ R⁹.

35. The method of claim 29, wherein the tyrosine kinase inhibitor comprises at least one of erlotinib or gefitinib.

36. A method for inhibiting MDP-induced, NFκB activation in a NOD2-bearing cell, the method comprising:
administering to the NOD2-bearing cell an amount of at least one tyrosine kinase inhibitor that inhibits nucleotide-binding oligomerization domain containing 2 (NOD2):receptor-interacting protein 2 (RIP2) signaling in the NOD2-bearing cell, wherein the amount is not cytotoxic to the NOD2-bearing cell.

37. The method of claim 36, the NOD2-bearing cell comprising a macrophage or colonic epithelial cell.

38. The method of claim 36, the tyrosine kinase inhibitor inhibiting phosphorylation of Y474 RIP2 tyrosine of a NOD2:RIP signaling complex.

39. The method of claim 36, tyrosine kinase inhibitor comprising an epidermal growth factor receptor (EGFR) inhibitor.

40. The method of claim 39, the EGFR inhibitor comprising a quinazoline derivative having the following general formula: 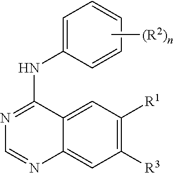
wherein
n is 1, 2 or 3 and each R² is independently halogeno, trifluoromethyl or (1-4C)alkyl;
R³ is (1-4C)alkoxy; and
R¹ is di-[(1-4C)alkyl]amino-(2-4C)alkoxy, pyrrolidin-1-yl-(2-4C)alkoxy, piperidino-(2-4C)alkoxy, morpholino-(2-4C)alkoxy, piperazin-1-yl-(2-4C)alkoxy, 4-(1-4C)alkylpiperazin-1-yl-(2-4C)alkoxy, imidazol-1-yl-(2-4C)alkoxy, di-[(1-4C)alkoxy-(2-4C)alkyl]amino-(2-4C)alkoxy, thiamorpholino-(2-4C)alkoxy, 1-oxothiamorpholino-(2-4C)alkoxy or 1,1-dioxothiamorpholino-(2-4C)alkoxy,
and wherein any of the above-mentioned R¹ substituents comprising a CH₂ (methylene) group which is not attached to a N or O atom optionally bears on said CH₂ group a hydroxy substituent;
or a pharmaceutically-acceptable salt thereof.

41. The method of claim 39, the EGFR inhibitor comprising a quinazoline derivative having the following general formula: 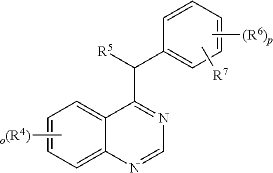
and to pharmaceutically acceptable salts and prodrugs thereof,
wherein:
o is 1, 2, or 3;
each R⁴ is independently selected from the group consisting of hydrogen, halo, hydroxy, hydroxyamino, carboxy, nitro, guanidino, ureido, cyano, trifluoromethyl, and —(C₁-C₄ alkylene)-W-(phenyl) wherein W is a single bond, O, S or NH;
or each R⁴ is independently selected from R¹² and (C₁-C₄)-alkyl substituted by cyano, wherein R¹² is selected from the group consisting of R⁸, —OR⁹, —NR⁹ R⁹, —C(O)R¹⁰, —NHOR⁸, —OC(O)R⁹, cyano, A and —YR⁸; R⁸ is C₁-C₄ alkyl; R⁹ is independently hydrogen or R⁸; R¹⁰ is —OR⁹ or —NR⁹ R⁹; A is selected from piperidino, morpholino, pyrrolidino, 4-R⁹-piperazin-1-yl, imidazol-1-yl, 4-pyridon-1-yl, —(C₁-C₄ alkylene)(CO₂ H), phenoxy, phenyl, phenylsulfanyl, C₂-C₄ alkenyl, and —(C₁-C₄ alkylene)C(O)NR⁹ R⁹; and Y is S, SO, or SO₂; wherein the alkyl moieties in R⁸, —OR⁹ and —NR⁹ R⁹ are optionally substituted by one to three substituents independently selected from halo and R¹², and wherein the alkyl moieties of said optional substituents are optionally substituted by halo or R¹², with the proviso that two heteroatoms are not attached to the same carbon atom, and with the further proviso that no more than three R¹² groups may comprise a single R⁴ group;
or each R⁴ is independently selected from —NHSO₂ R⁸, phthalimido-(C₁-C₄)-alkylsulfonylamino, benzamido, benzenesulfonylamino, 3-phenylureido, 2-oxopyrrolidin-1-yl, 2,5-dioxopyrrolidin-1-yl, and R¹³—(C₂-C₄)-alkanoylamino wherein R¹³ is selected from halo, —OR⁹, C₂-C₄ alkanoyloxy, —C(O)R¹⁰, and —NR⁹ R⁹; and wherein the foregoing R⁴ groups are optionally substituted by 1 or 2 substituents independently selected from halo, C₁-C₄ alkyl, cyano, methanesulfonyl and C₁-C₄ alkoxy;
or two R⁴ groups are taken together with the carbons to which they are attached to form a 5-8 membered ring that includes 1 or 2 heteroatoms selected from O, S and N;
R⁵ is hydrogen or C₁-C₆ alkyl optionally substituted by 1 to 3 substituents independently selected from halo, C₁-C₄ alkoxy, —NR⁹ R⁹, and —SO₂ R⁸;
p is 1 or 2 and each R³ is independently selected from hydrogen, halo, hydroxy, C₁-C₆ alkyl, —NR⁹ R⁹, and C₁-C₄ alkoxy, wherein the alkyl moieties of said R⁶ groups are optionally substituted by 1 to 3 substituents independently selected from halo, C₁-C₄ alkoxy, —NR⁹ R⁹, and —SO₂ R⁸; and,
R⁷ is azido or -(ethynyl)-R¹⁴ wherein R¹⁴ is hydrogen or C₁-C₆ alkyl optionally substituted by hydroxy, —OR⁹, or —NR⁹ R⁹.

42. The method of claim 36, wherein the tyrosine kinase inhibitor comprises at least one of erlotinib or gefitinib.


SEQUENCE LISTING (9 sequences)

SEQ ID NO: 1 - DNA, 21 bp, Homo sapiens
cacgggcgagtttactatgta

SEQ ID NO: 2 - DNA, 21 bp, Homo sapiens
caagagctatgagcaactgaa

SEQ ID NO: 3 - DNA, 21 bp, Homo sapiens
atgggtagcctcaccatgaaa

SEQ ID NO: 4 - DNA, 21 bp, Homo sapiens
tgccgccgacaaatacaaata

SEQ ID NO: 5 - DNA, 21 bp, Homo sapiens
aagaagaaatgtgtttcataa

SEQ ID NO: 6 - DNA, 20 bp, Homo sapiens
tccacaagcgccttcggtcc

SEQ ID NO: 7 - DNA, 20 bp, Homo sapiens
gtggctgtctgtgtggggcg

SEQ ID NO: 8 - DNA, 18 bp, Homo sapiens
gacctgacctgccgtcta

SEQ ID NO: 9 - DNA, 21 bp, Homo sapiens
gttgctgtagccaaattcgtt